(19) United States
(12) Patent Application Publication
(10) Pub. No.: US 2024/0270842 A1
(43) Pub. Date: Aug. 15, 2024

(54) USE OF AN ANTI-CD19 ANTIBODY TO TREAT MYASTHENIA GRAVIS

(71) Applicant: Viela Bio, Inc, Deerfield, IL (US)

(72) Inventors: Eliezer KATZ, Durham, NC (US); John Nolan RATCHFORD, Lutherville, MD (US); Liron ABUHSIRA, Clarksburg, MD (US)

(21) Appl. No.: 18/559,195

(22) PCT Filed: May 6, 2022
(86) PCT No.: PCT/US2022/028063

§ 371 Date: Nov. 6, 2023

Related U.S. Application Data
(60) Provisional application No. 63/185,613, filed on May 7, 2021.
(60) Provisional application No. 63/303,655, filed on Jan. 27, 2022.

(51) Int. Cl. C07K 16/28; A61K 9/00; A61K 31/365; A61K 31/436; A61K 31/52; A61K 31/5377; A61K 31/573; A61K 39/395; A61P 37/06

(52) U.S. Cl. CPC C07K 16/2803; A61K 9/0019; A61K 31/365; A61K 31/436; A61K 31/52; A61K 31/5377; A61K 31/573; A61K 39/3955; A61P 37/06; C07K 2317/24

(57) ABSTRACT

Methods for using an anti-CD19 antibody to treat autoimmune disease are disclosed herein. In particular, the disclosure provides for the use of Inebilizumab, a humanized, affinity-optimized, afucosylated IgG1 kappa monoclonal antibody to treat myasthenia gravis.

### CROSS-REFERENCE TO RELATED APPLICATIONS

This application claims priority to U.S. Provisional Patent Application No. 63/185,613, filed May 7, 2021, and U.S. Provisional Patent Application No. 63/303,655, filed Jan. 27, 2022, each of which is entirely incorporated herein by reference for all purposes. DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: HOPA-032_02WO_SeqList_ST25, date recorded May 5, 2022, file size 12,288 bytes).

### TECHNICAL FIELD

The present disclosure is related to compositions and methods of treating myasthenia gravis comprising administering an anti-CD19 antibody to a subject in need.

### BACKGROUND

Myasthenia gravis (MG) is a rare autoimmune disorder, caused by binding of antibodies to the acetylcholine receptor (AChR) or functionally related molecules on the postsynaptic neuromuscular junction. Incidence ranges from 0.3-2.8 per 100,000 and it is estimated that more than 700,000 people worldwide are affected by MG (Sanders D B, Wolfe G I, Benatar M, Evoli A, Gilhus N E, Illa I, et al. International consensus guidance for management of myasthenia gravis. Executive Summary. Neurology 2016; 87:419-25). There are reports of increasing incidence of MG, which may in part be attributed to improved diagnostics but may also indicate a role of environmental factors (Gilhus N E, Tzartos S, Evoli A, Palace J, Burns T M, Verschuuren JJGM. Myasthenia gravis. Nat Rev Dis Primers 2019; 5:30; Yi J S, Guptill J T, Stathopoulos P, Nowak R J, O'Connor K C. B cells in the pathophysiology of myasthenia gravis. Muscle Nerve 2018; 57:172-84).

Approximately 85% of subjects with MG are categorized as having generalized disease, with common symptoms including ptosis, diplopia, dyspnea, and generalized muscle weakness. Conversely, in approximately 15% of subjects with MG, symptoms are restricted to the ocular muscles (Gilhus N E, Tzartos S, Evoli A, Palace J, Burns T M, Verschuuren JJGM. Myasthenia gravis. Nat Rev Dis Primers 2019; 5:30).

Several case series report that B-cell depletion with an anti-CD20 monoclonal antibody (mAb) can reduce disease severity in the treatment of refractory generalized myasthenia gravis (MG) (Iorio R, Damato V, Alboini P E, Evoli A. Efficacy and safety of rituximab for myasthenia gravis: a systematic review and meta-analysis. J Neurol 2015; 262:1115-9). However, not all studies have found a benefit of anti-CD20 therapy in MG. Inebilizumab is a humanized mAb that depletes CD19+ B cells. CD19 expression persists on late-stage, antibody-secreting B cells (plasmablasts and some plasma cells) after CD20 expression has been lost, which may be important in diseases driven by pathogenic autoantibodies.

Anticholinesterases and immunosuppressive therapies (ISTs) are the standard of care for the medical treatment and have notably reduced mortality and improved quality of life (QOL) for subjects with MG. Approximately 85-90% of subjects with MG respond to standard of care therapies (Urban P P, Jacobi C, Jander S. treatment standards and individualized therapy of myasthenia gravis. Neurology International Open 2018; 2: E84-92). However, onset of efficacy may be slow, durability of efficacy may be limited, and side effects may occur with long-term use for some ISTs. Additionally, a subgroup (˜10-20%) of subjects with MG do not respond to multiple combinations of drugs and are referred to as being refractory subjects. Therefore, there is a need to develop new therapeutics for MG that have rapid onset of action, good treatment durability, and which can help refractory subjects. Oral corticosteroids are also commonly used for MG and are associated with considerable side effects; reduction in the need for oral corticosteroids is therefore another important goal of MG treatment.

VIB551 is a humanized, affinity-optimized, afucosylated IgG1 kappa monoclonal antibody that binds to the B-cell surface antigen CD19. In contrast to anti-CD20 monoclonal antibodies that recognize and deplete a subset of CD20-expressing T lymphocytes (in addition to B lymphocytes; Palanichamy A, Jahn S, Nickles D, et al. Rituximab efficiently depletes increased CD20-expressing T cells in multiple sclerosis subjects. J Immunol 2014; 193: 580-6.), anti-CD19 antibodies recognize and deplete lymphocytes exclusively from the B-cell lineage.

### BRIEF SUMMARY

Provided are methods of treating myasthenia gravis (MG). The methods comprising administering to a subject in need of treatment for MG an antibody that comprises a heavy chain variable region (VH) comprising the amino acid of SEQ ID NO:1, and a light chain variable region (VL) comprising the amino acid of SEQ ID NO: 2, wherein the antibody is administered intravenously at a dose of from about 250 mg to about 350 mg every 6 months, thereby treating the MG. In aspects, two weeks prior to the administering, an initial dose of the antibody is administered to the subject, wherein the initial dose is from about 250 mg to about 350 mg. In aspects, a second dose of the antibody of about 300 mg is administered two weeks after the administering. In aspects, the administering is effective in (i) depleting at least about 90% of circulating CD20+ B cells for at least six months; (ii) does not increase incidence of an infection in the subject; or (iii) (i) and (ii). In aspects, the administering reduces a level of peripheral blood CD20⁻ plasmablasts and plasma cells within about 8 days following the administering. In aspects, the dose of a provided composition is about 300 mg. In aspects, the administering of a provided composition is effective in reducing MG-related disability. In aspects, the administering of a provided composition is effective in reducing frequency of MG exacerbations. In aspects, a subject is acetylcholine receptor antibody positive (AChR-Ab+). In aspects, a subject is muscle-specific kinase-antibody positive (MuSK-Ab+). In aspects, a subject is acetylcholine receptor antibody positive (AChR-Ab+) and muscle-specific kinase-antibody positive (MuSK-Ab+). In aspects, the frequency of MG exacerbations is reduced by at least about 1-fold as compared to an otherwise comparable subject lacking the administering. In aspects, an administering is effective in reducing fatigue in a subject as determined by Neuro-QoL Fatigue score. subject is further administered one or more additional therapies. In aspects, one or more additional therapies comprise one or more standard-of-care therapies. In aspects, one or more standard-of-care therapies comprises a corticosteroid, a nonsteroidal immunosuppressive therapy, or both. In aspects, the one or more standard-of-care therapies comprises the corticosteroid, and wherein the corticosteroid comprises prednisone. In aspects, the one or more additional therapies are one or more of azathioprine, mycophenolate mofetil, mycophenolic acid, or tacrolimus. In aspects, an antibody is Inebilizumab. In aspects, an antibody is administered at a dose of about 300 mg. In aspects, an administration, treats MG as determined by: a) a reduction in MG activities of daily living score; b) a reduction of a MG score; c) an increase in a quality-of-life score; and/or d) a reduction in incidence of exacerbations. In aspects, MG is refractory MG. In aspects, a subject in need of treatment for MG has uncontrolled MG, on a standard-of-care therapy, as determined by one or more of: a) a MG Foundation of America classification of II, III, or IV; b) an MG activities of daily living (MG-ADL) score≥6, with >50% of this score being attributed to non-ocular items; or c) a quantitative MG (QMG) score≥11. In aspects, when a subject in need of treatment for MG is receiving a dosage of prednisone over 5 mg/day or equivalent, the subject will undergo tapering of the prednisone dosage to 5 mg/day. In aspects, a maximum dosage of: a) tacrolimus≤3 mg/day; b) azathioprine≤3 mg/kg/day; c) mycophenolate mofetil≤3 g/day; and/or d) mycophenolic acid≤1440 mg/day. In aspects, a subject in need of treatment for MG has uncontrolled MG, on a standard-of-care therapy, as determined by the MG-ADL score of ≥6, with >50% of the score being attributed to non-ocular items, and wherein the MG-ADK score is reduced after the administering. In aspects, the reduction is of at least about 2 points. In aspects, a method comprises determining: a) a B-cell subset phenotype; b) a B-cell receptor repertoire; c) a B-cell gene expression profiling; or d) any combination thereof. In aspects, an administration is effective in reducing or eliminating mature plasma cells in the subject. In aspects, an administration results in longer-lasting B-cell reduction or elimination as compared to an otherwise comparable method comprising administration of an anti-CD20 therapy.

Provided are methods of treating myasthenia gravis (MG), comprising: (a) administering a standard-of-care therapy in an amount sufficient to treat MG in a subject in need thereof; and (b) administering a pharmaceutical composition that comprises an antibody that comprises a heavy chain variable region (VH) comprising the amino acid of SEQ ID NO:1, and a light chain variable region (VL) comprising the amino acid of SEQ ID NO: 2 at a dose of from about 250 mg to about 350 mg. In aspects, the standard-of-care therapy comprises a corticosteroid. In aspects, the standard-of-care therapy comprises a nonsteroidal immunosuppressive therapy. In aspects, the standard-of-care therapy comprises a corticosteroid and a nonsteroidal immunosuppressive therapy. In aspects, the corticosteroid comprises prednisone. In aspects, the antibody is Inebilizumab. In aspects, the dose is from about 275 mg to about 325 mg, from about 290 mg to about 310 mg, or 205 mg to about 305 mg. In aspects, the dose is about 300 mg. In aspects, the subject has a CD19+ B-cell count≥40 cells/μL prior to administration of the antibody. In aspects, the subject has a myasthenia gravis foundation of America (MGFA) clinical classification selected from class II, class III, or class IV. In aspects, the subject has anti-AChR antibodies or anti-MuSK antibodies, or both. In aspects, the subject does not have an immunodeficiency disorder.

Provided are methods of treating myasthenia gravis (MG), comprising: administering Inebilizumab to a subject in need of treatment for MG, wherein the Inebilizumab is administered intravenously at a dose of about 300 mg every 6 months. In aspects, two weeks prior to administering the 300 mg Inebilizumab every 6 months, an initial 300 mg Inebilizumab dose is administered to the subject. In aspects, provided can be a method comprising: administering Inebilizumab to a subject diagnosed with MG, wherein the Inebilizumab is administered intravenously at a first dose of 300 mg, a second dose of 300 mg two weeks after the first dose, and subsequent doses of 300 mg every 6 months following the first dose.

Provided are methods of treating a subject diagnosed with MG, the methods comprising: administering Inebilizumab to a subject diagnosed with MG, wherein the Inebilizumab is administered at a dose that: (i) depletes at least 90% of circulating CD20+ B cells for at least six months; and (ii) does not increase incidence of an infection in the subject. In aspects, the Inebilizumab further depletes peripheral blood CD20⁻ plasmablasts and plasma cells within about 8 days following the administering. In aspects, the dose is 300 mg.

Provided are methods of reducing MG-related disability, comprising: administering Inebilizumab to a subject in need of treatment for MG, wherein the Inebilizumab is administered intravenously at a first dose of 300 mg, a second dose of 300 mg two weeks after the first dose, and subsequent doses of 300 mg every 6 months following the first initial dose.

Provided are methods of reducing the frequency of MG exacerbations, comprising: administering Inebilizumab to a subject in need of treatment for MG, wherein the Inebilizumab is administered intravenously with at a first dose of 300 mg, a second dose of 300 mg two weeks after the first dose, and subsequent doses of 300 mg every 6 months following the first dose.

In aspects of any of the methods, a subject is acetylcholine receptor antibody positive (AChR-Ab+). In aspects, the subject is muscle-specific kinase-antibody positive (MuSK-Ab+). In aspects, the subject is acetylcholine receptor antibody positive (AChR-Ab+) and muscle-specific kinase-antibody positive (MuSK-Ab+). In aspects, administration of the Inebilizumab reduces the frequency of MG exacerbations. In aspects, administration of the Inebilizumab reduces fatigue as determined by Neuro-QoL Fatigue score. In aspects, the subject is further administered one or more additional therapies. In aspects, one or more additional therapies comprise one or more standard-of-care therapies. In aspects, standard-of-care therapy comprises a corticosteroid, a nonsteroidal immunosuppressive therapy, or both. In aspects, the corticosteroid is prednisone. In aspects, the one or more additional therapies are one or more of azathioprine, mycophenolate mofetil, mycophenolic acid, or tacrolimus. In aspects, the Inebilizumab comprises a heavy chain variable region (VH) comprising the amino acid of SEQ ID NO:1, and a light chain variable region (VL) comprising the amino acid of SEQ ID NO: 2. In aspects, the Inebilizumab is administered intravenously. In aspects, administration of Inebilizumab treats the MG as determined by: a) a reduction in MG activities of daily living score; b) a reduction of a MG score; c) an increase in a quality-of-life score; and/or d) a reduction in incidence of exacerbations. In aspects, the MG is refractory MG. In aspects, the subject in need of treatment for MG has uncontrolled MG, on a standard-of-care therapy, as determined by one or more of: a) a MG Foundation of America classification of II, III, or IV; b) an MG activities of daily living (MG-ADL) score≥6, with >50% of this score being attributed to non-ocular items; or c) a quantitative MG (QMG) score≥11. In aspects, if the subject in need of treatment for MG is receiving a dosage of prednisone over 5 mg/day or equivalent, the subject will undergo tapering of the prednisone dosage to 5 mg/day. In aspects, a maximum dosage of: a) tacrolimus is about ≤3 mg/day; b) azathioprine is about ≤3 mg/kg/day; c) mycophenolate mofetil is about ≤3 g/day; and/or d) mycophenolic acid is about ≤1440 mg/day. In aspects, the MG-ADK score is reduced after the administration of Inebilizumab. In aspects, the reduction is of at least about 2 points. In aspects, a method comprises determining: a) B-cell subset phenotype; b) B-cell receptor repertoire; c) B-cell gene expression profiling; or d) any combination thereof. In aspects, administering of a composition provided herein is effective in reducing or eliminating mature plasma cells in the subject. In aspects, administration results in longer-lasting B-cell reduction or elimination as compared to an otherwise comparable method comprising administration of an anti-CD20 therapy.

The description provides for methods of treating MG in a subject in need of treatment for MG, treating a subject diagnosed with MG, reducing MG-related disability in a subject need of treatment for MG and reducing the frequency of MG exacerbations in a subject need of treatment for MG.

In aspects, the disclosure provides a method of treating MG. In the method, VIB551 is administered to a subject in need of treatment for MG. The VIB551 is administered intravenously at a dose of 300 mg every 6 months. In aspects of the method, an initial 300 mg dose VIB551 is administered two weeks prior to the 300 mg VIB551 dose being administered every 6 months.

In aspects, the disclosure provides a method of treating a subject diagnosed with MG. In one such method, VIB551 is administered to a subject diagnosed with MG. The VIB551 is administered intravenously with a first initial dose of 300 mg, a second initial dose of 300 mg two weeks after the first initial dose, and subsequent doses of 300 mg every 6 months following the first initial dose. In another such method of treating a subject diagnosed with MG in which VIB551 is administered to a subject diagnosed with MG, the VIB551 is administered at a dose that: (i) depletes at least 90% of circulating CD20+ B cells for at least six months, and (ii) does not increase risk of infections in the subject. In aspects of the methods of treating a subject diagnosed with MG, the VIB551 further depletes peripheral blood CD20 plasmablasts and plasma cells within 8 days following the administering. In yet other aspects, the dose VIB551 is 300 mg. In further yet aspects, the VIB551 is administered intravenously.

In aspects, the disclosure provides a method of reducing MG-related disability. In the method, VIB551 is administered to a subject in need of treatment for MG. The VIB551 is administered intravenously with a first initial dose of 300 mg, a second initial dose of 300 mg two weeks after the first initial dose, and subsequent doses of 300 mg every 6 months following the first initial dose.

In aspects, the disclosure provides a method of reducing the frequency of MG exacerbations. In the method, VIB551 is administered to a subject in need of treatment for MG. The VIB551 is administered intravenously with a first initial dose of 300 mg, a second initial dose of 300 mg two weeks after the first initial dose, and subsequent doses of 300 mg every 6 months following the first initial dose

In aspects of any of the methods provided herein, the subject is acetylcholine receptor antibody positive (AChR-Ab+). In aspects of any of the methods provided herein, the subject is muscle-specific kinase-antibody positive (MuSK-Ab+). In aspects of any of the methods provided herein, the subject is acetylcholine receptor antibody positive (AChR-Ab+) and muscle-specific kinase-antibody positive (MuSK-Ab+). In aspects of any of the methods provided herein, administration of the VIB551 reduces the frequency of MG exacerbations. In aspects of any of the methods provided herein, administration of the VIB551 reduces fatigue.

In aspects, the subject is further administered one or more additional therapies. In aspects in which the subject is further administered one or more additional therapies, the one or more additional therapies are one or more standard of care therapies. In aspects, aspects in which the one or more additional therapies are one or more standard of care therapies, the additional therapy is a corticosteroid, which may be prednisone. In aspects in which the subject is further administered one or more additional therapies, the one or more additional therapies are one or more of azathioprine, mycophenolate mofetil, mycophenolic acid, or tacrolimus.

In aspects of any of the methods provided herein, the VIB551 has a heavy chain variable region that includes the amino acid sequence of SEQ ID NO:1 and a light chain variable region that includes the amino acid of SEQ ID NO:2. In aspects of any of the methods provided herein, the administration of the VIB551 treats the MG. In aspects of any of the methods provided herein, the MG is refractory MG.

### BRIEF DESCRIPTION OF FIGURES

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts an exemplary study flow diagram.

FIG. 2 depicts the design scheme for AChR-Ab+ population. AChR-Ab+=the population of subjects who are antibody positive for the acetylcholine receptor; CS=corticosteroid; D=Day; IV=intravenous(ly); MG=Myasthenia Gravis; MG-ADL=Myasthenia Gravis Activity of Daily Living score; MGFA=Myasthenia Gravis Foundation of America; OLP=Open-Label Period; QMG=Quantitative Myasthenia Gravis scale; RCP=Randomized Controlled Period; SoC=Standard of Care.

FIG. 3 depicts the design scheme for MuSK-Ab+ population. CS=corticosteroid; D=Day; IV=intravenous(ly); MG=Myasthenia Gravis; MG-ADL=Myasthenia Gravis Activity of Daily Living score; MGFA=Myasthenia Gravis Foundation of America classification; MuSK-Ab+=the population of subjects who are antibody positive for muscle-specific kinase; OLP=Open-Label Period; QMG=Quantitative Myasthenia Gravis scale; RCP=Randomized Controlled Period; SoC=standard of care.

FIG. 4 depicts the VH (SEQ ID NO:1) and VL (SEQ ID NO:2) amino acid sequences of the VIB551 antibody.

FIG. 5A-FIG. 5C are graphics illustrating a possible Inebilizumab mechanism of action in MG. FIG. 5A shows that Inebilizumab targets the CD19 receptor, consequently inhibiting autoantibody production and depleting circulating B cells. FIG. 5B shows that autoantibodies disrupt the normal architecture and functioning of the neuromuscular junction in MG. FIG. 5C shows that Anti-AChR autoantibodies drive MG by: 1) complement activation and muscle membrane damage; 2) AChR crosslinking and internalization; and 3) direct blockade of receptors. Anti-MuSK autoantibodies drive MG via disruption of MuSK/LRP4 interaction, which interferes with AChR clustering. ACh, acetylcholine; AChR, acetylcholine receptor, anti-AChR Ab, anti-acetylcholine receptor antibody; LRP4, LDL-receptor related protein 4; MuSK, muscle-specific tyrosine kinase; NMJ, neuromuscular junction.

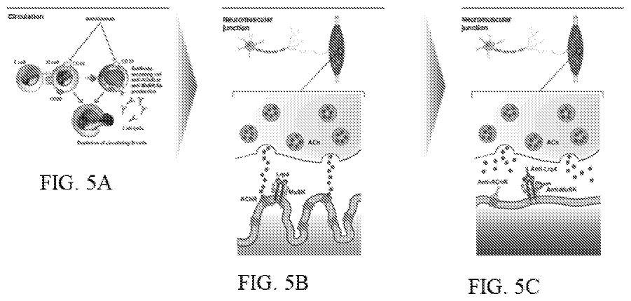

### DETAILED DESCRIPTION

Described herein are compositions that comprise VIB551 (also referred to HZN551, MEDI551, UPLIZNA™ or Inebilizumab) and its usefulness in methods for treating myasthenia gravis (MG), in methods for reducing MG-related disability, in methods for reducing the frequency of MG exacerbations, and in methods for improving MG-related quality of life. In aspects, the disclosure provides a method of treating myasthenia gravis, the method comprising: administering VIB551 to a subject in need of treatment for MG, wherein the VIB551 is administered intravenously at a dose of 300 mg every 6 months. Provided is also a method of utilizing VIB551 to treat MG in subjects with inadequately controlled disease on one or more standard-of-care therapies.

### Definitions

Unless defined otherwise, all technical and scientific terms used herein have the same meaning as commonly understood by one of ordinary skill in the art to which the subject matter pertains. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

As used in this specification and the appended claims, the singular forms “a,” “an” and “the” include plural referents unless the context clearly dictates otherwise.

Ranges can be expressed herein as from “about” one particular value, and/or to “about” another particular value. When such a range is expressed, aspects include from the one particular value and/or to the other particular value. Similarly, when values are expressed as approximations, by use of the antecedent “about,” it will be understood that the particular value forms aspects. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5. The term “about” also accounts for typical error or imprecision in measurement of values.

As used herein, the term “subject” refers to any individual, e.g., a human or a non-human mammal, for whom diagnosis, prognosis, or therapy is desired. The term “subject” may mean a human or non-human mammal affected, likely to be affected, or suspected to be affected with a disease. The terms “subject” and “patient” are used interchangeably herein. In aspects, the subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile, fish, or the like.

As used herein, the term “a subject in need thereof” includes subjects that could or would benefit from the methods described herein. Subjects in need of treatment include, without limitation, those already with the condition or disorder, those prone to having the condition or disorder, those in which the condition or disorder is suspected, as well as those in which the condition or disorder is to be prevented, ameliorated, or reversed.

As used herein, the term “normal subject” refers to any healthy individual, e.g., a human or a non-human mammal, not affected with any disease or suspected of being affected with a disease or condition.

As used herein, “treating” or “treat” describes the management and care of a subject for the purpose of combating a disease, condition, or disorder and includes the administration of VIB551 used in the methods described herein to alleviate the symptoms or complications of a disease, condition, or disorder, or to eliminate the disease, condition, or disorder. Thus, the term “treat” or “treating” refers to both therapeutic measures and prophylactic or preventative measures, wherein the objective is to prevent, slow down (lessen), or ameliorate the progression of a disease (e.g., MG). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total). The term “treat” can also include treatment of a cell in vitro or an animal model.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

Generalized myasthenia gravis (MG) is a chronic autoimmune disorder characterized at least by ocular, bulbar, respiratory, and limb muscle weakness. The disease is driven, in part, by autoantibodies reactive against components of the neuromuscular junction (NMJ). Standard-of-care therapies comprising thymectomy, monoclonal antibody therapy, anticholinesterase therapy, immunosuppressive drugs, plasmapheresis, and intravenous immunoglobulin can be ineffective at controlling disease and are associated with various toxicities. MG is most often treated with a combination of cholinesterase inhibitors and immunosuppressive drugs. Cholinesterase inhibitor therapy is limited and may even be detrimental in the treatment of subjects having anti-muscle-specific tyrosine kinase (MuSK) antibody related MG, most likely because of low acetylcholinesterase concentrations at the neuromuscular junction (NMJ). Further, corticosteroid-sparing immunosuppressive agents such as azathioprine, mycophenolate, cyclosporine, tacrolimus, and others, may be added to treatment regimens; however, clinical response varies, as they have a slower onset of action compared with corticosteroids, and carry the risk of adverse events such as myelosuppression and hepatotoxicity. Accordingly, there remains a need for effective treatment that addresses the limitations of current standard-of-care therapies.

### Inebilizumab

The pathophysiology of MG is driven, in part, by B-cell-mediated immunity and the production of specific antibodies. Accordingly, provided herein are compositions that comprise Inebilizumab and methods of using the same for the treatment of MG. In aspects, a subject has anti-acetylcholine receptor (AChR) or anti-muscle-specific tyrosine kinase (MuSK) antibodies, AChR-Ab⁺ or MuSK-Ab⁺ respectively.

Approximately 85% of subjects with MG have detectable antibodies against AChR and approximately 7% have detectable antibodies against muscle-specific kinase (MuSK) (Hehir and Silvestri, 2018). Antibodies in acetylcholine receptor antibody positive (AChR-Ab+) MG are of IgG1 and IgG3 isotypes and are therefore capable of activating complement. Clinical symptoms are caused by (1) direct blockade of acetylcholine binding sites, (2) crosslinking of AChRs by divalent antibodies, which increases receptor endocytosis and degradation, and (3) complement mediated damage of the entire muscle endplate. Antibodies in MuSK antibody positive (MuSK-Ab+) MG are predominantly of the IgG4 isotype and are therefore unable to bind factor C1q and cannot cause complement activation. There are similarities between AChR Ab+MG and MuSK-Ab+MG, although involvement of neck, shoulder, facial, and bulbar muscles is more common in MuSK-Ab+ disease (Ha and Richman, 2015). Treatment response diverges between these subgroups, with MuSK Ab+ subjects being less responsive to anticholinesterase agents and more likely to remain steroid dependent compared with AChR Ab+ subjects.

VIB551 and methods of making thereof are described in International PCT Patent Application PCT/US2007/077916, published as WO 2008/031056, which is hereby incorporated by reference (PCT/US2007/077916 refers to VIB551 as “16C4”). In aspects, VIB551 (also referred to HZN551, MEDI551, UPLIZNA™ or Inebilizumab; disclosed in U.S. application Ser. No. 11/852,106 and Int'l Appl. No. PCT/US20/29613, which are incorporated herein by reference in their entireties) is administered in any of the methods disclosed herein.

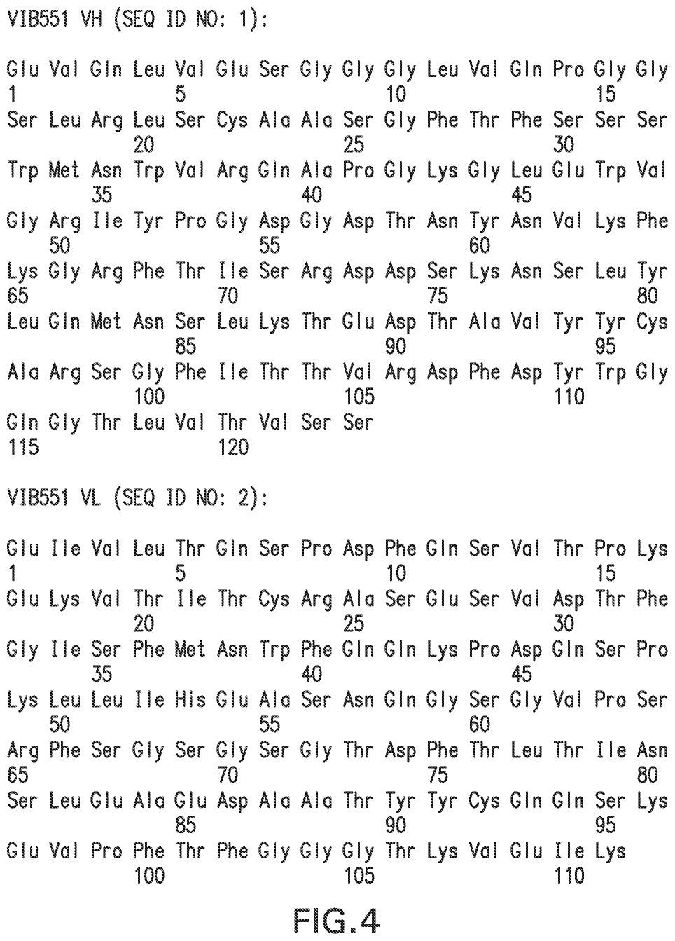

In aspects, VIB551 may have the VH amino acid sequence and a VL amino acid sequence as shown in FIG. 4. In aspects, the VIB551 may comprise a heavy chain variable region (VH) comprising the amino acid of SEQ ID NO:1 and a light chain variable region (VL) comprising the amino acid of SEQ ID NO: 2. The VIB551 administered in the methods may have the VH amino acid sequence and the VL amino acid sequence as shown in FIG. 4, but for one or more changes in amino acid residues that do not alter the function of the VIB551 amino acid sequence. The number of amino acid changes may be 1 amino acid residue change, 2 amino acid residue changes, 3 amino acid residue changes, 4 amino acid residue changes, or 5 amino acid residue changes. In aspects, the VIB551 used in the methods disclosed herein is at least about 90%, about 91%, about 92%, about 93%, about 94%, about 95%, about 96%, about 97%, about 98%, about 99% or identical to the VH and VL sequences disclosed in FIG. 4. The VIB551 administered in the methods may have the CDR amino acid sequences of the VH and the VL sequences as shown in FIG. 4 but may have one or more alterations in the framework regions of the VH and the VL sequences shown in FIG. 4. The amino acid sequence of each CDR of VIB551 are as follows: VH CDR1 (SEQ ID NO:3), VH CDR2 (SEQ ID NO:4), VH CDR3 (SEQ ID NO:5), VL CDR1 (SEQ ID NO:6), VL CDR2 (SEQ ID NO:7) and VL CDR3 (SEQ ID NO:8). In aspects, the VIB551 may comprise a heavy chain comprising the amino acid of SEQ ID NO:9 and a light chain comprising the amino acid of SEQ ID NO:10. The VIB551 administered in the methods may have the heavy chain amino acid sequence of SEQ ID NO:9 and the light chain amino acid sequence of SEQ ID NO:10 but for one or more changes in amino acid residues that do not alter the function of VIB551. The number of amino acid changes may be 1 amino acid residue change, 2 amino acid residue changes, 3 amino acid residue changes, 4 amino acid residue changes, or 5 amino acid residue changes.

In aspects, another anti-CD19 antibody may be substituted to a subject in any of the methods disclosed herein. In aspects, the other anti-CD19 antibody, if other than VIB551, may be any of, for example, such as CD19 antibody, e.g., MOR00208 (also referred to as Xmab 5574 or tafasitamab; disclosed in U.S. Patent Application No. 20170137516), an anti-CD20 antibody, e.g., Rituximab (antibody C2B8 in WO94/11026), Ocrelizumab (also referred to as Ocrevus® or PRO70769; disclosed in Vugmeyster, Y., et al., J. Immunother. 28(2005):212-219, WO 2004/056312 and WO 2006/084264), Ofatumumab (also referred to as HuMax-CD20 or Azerra®; disclosed as antibody 2F2 in WO04/35607) or Obinutuzumab (also referred to as Gazyva®; disclosed in WO2017/148880); an anti-CD22 antibody, e.g., Epratuzumab (antibody hLL2 in U.S. Pat. No. 5,789,554); or a BLyS inhibitor, e.g., Belimumab (also referred to as Lymphostat-B; disclosed in WO 02/02641), BR3-Fc (disclosed in WO 05/00351), AMG-623 (also referred to as blisibimod; PubChem SID: 163312341), or Atacicept (U.S. Patent Application Publication No. 20060034852).

The VIB551 administered in the methods disclosed herein may be packaged in a 10-mL vial filled with a nominal 10-mL solution of VIB551 at a concentration of 10 mg/mL, containing 20 mM histidine/histidine hydrochloride, 70 mM NaCl, 106 mM (4% [w/v]) trehalose dehydrate, and 0.01% (w/v) polysorbate 80, pH 6.0. Provided are also kits or containers that comprise the VIB551 of the disclosure and instructions for utilizing the same.

### Method of Treatment

Provided herein are methods of using Inebilizumab for the treatment of MG. Inebilizumab can be administered at any dose. In aspects, a subject may also receive a pre-conditioning treatment ahead of treatment with Inebilizumab. In aspects, a subject may also undergo tapering of a therapeutic ahead of treatment with Inebilizumab. Accordingly, various therapeutic regimens are contemplated that comprise Inebilizumab.

In aspects, the methods disclosed herein are used to treat subjects with MG who are AChR Ab+. In aspects, the methods disclosed herein are used to treat subjects with MG who are MuSK-Ab+. In aspects, the methods disclosed herein are used to treat subjects with MG who are AChR Ab+ and MuSK-Ab+. In aspects, a method comprises determining the serostatus of a subject. Serostatus may be evaluated before, during, or after treatment with Inebilizumab.

In aspects, provided are methods of treatment a subject with inadequately controlled disease on standard-of-care therapy. Exemplary standard-of-care therapies are described herein and include but are not limited to corticosteroids and nonsteroidal immunosuppressive therapy. In aspects, a subject is treated that comprises inadequate symptom control with sufficient disease severity to warrant additional treatment as determined by an attending physician. Inadequate symptom control can also be determined as having one or more of: (a) an MG Foundation of America classification of II, III, or IV; (b) an MG activities of daily living (MG-ADL) score≥6 with >50% of this score being attributed to non-ocular items; or (c) a quantitative MG (QMG) score≥11.

In aspects, provided are methods that comprise tapering of a therapeutic. A therapeutic may be tapered ahead of Inebilizumab administration, concurrent with Inebilizumab administration, or after treatment with Inebilizumab. In aspects, a subject that may be treated with compositions of the disclosure is undergoing treatment with prednisone at about 40 mg/day or 80 mg every other day. In aspects, a subject that is treated with prednisone over 5 mg/day or equivalent may undergo steroid tapering until a dosage of 5 mg/day is reached. The tapering may begin at any point ahead, concurrent, or after administration of Inebilizumab.

The administration of a compositions that comprises Inebilizumab in the methods described herein may be every 6 months or approximately 6 months. For example, the administration of Inebilizumab or the derivative thereof may be every 6 months, every 180 days, between about every 170 and about every 190 days, between about every 175 and about every 185 days, between about every 175 and about every 190 days, or between about every 170 and about every 185 days. The administration of Inebilizumab may be about every 26 weeks, about every 25 weeks, about every 27 weeks, between about every 25 and about every 27 weeks, between about every 25 and about every 26 weeks, or between about every 26 and about every 27 weeks. Prior to administering Inebilizumab or the derivative thereof every six months in the methods described herein, an initial Inebilizumab or derivative of Inebilizumab dose may be administered to the subject. If an initial dose is administered, the initial dose may be administered approximately 2 weeks before the approximately every 6-month dosing. The administering of the initial dose approximately 2 weeks before every approximately 6-month dosing may be the administering of the initial dose 12 days, 13 days, 14 days, 15 days, or 16 days before the approximately every 6 months dosing. The initial dose may or may not be co-administered with oral corticosteroids.

The dose of Inebilizumab administered intravenously in the methods described herein may be 300 mg or approximately 300 mg. An approximately 300 mg dose may be a dose of about 250 mg to about 350 mg, it may be a dose of about 275 mg to about 325 mg, it may be a dose of about 290 mg to about 310 mg, it may be a dose of about 205 mg to about 305 mg, or it may be a dose of 275 mg, 280 mg, 285 mg, 290 mg, 295 mg, 300 mg, 305 mg, 310 mg, 315 mg, 320 mg, or 325 mg.

In aspects, a subject is administered Inebilizumab at a dose of about 300 mg on day 1 and day 15, followed by a 300 mg infusion at 6 months (day 183) for a subject having AChR-Ab+ serostatus. In aspects, a subject having the MuSK-Ab+ serostatus can be administered Inebilizumab on days 1 and 15 of a randomized control period. Following this treatment period, subjects can receive infusions of Inebilizumab on days 1 and 15, and at 6 months (day 183) after the start of the open label period. On day 15, participants who received Inebilizumab during the randomized control period can receive placebo, and those who received placebo during the randomized control period will receive Inebilizumab.

In aspects, the disclosure is directed to a method of reducing MG-related disability, the method comprising: administering VIB551 to a subject in need of treatment for MG. In aspects, the disclosure is directed to a method of reducing the frequency of MG exacerbations, the method comprising administering VIB551 to a subject in need of treatment for MG. In aspects, VIB551 treats MG by reducing the frequency of MG exacerbations. In aspects, the frequency of MG exacerbations is reduced by at least about: 5%, 10%, 20%, 30%, 40%, 50%, 60%, 70%, 80%, 90%, 100%, 120%, 150%, 200%, or about 400%. In aspects, the frequency of MG exacerbations is reduced by at least about: 1-fold, 5-fold, 10-fold, 20-fold, 30-fold, 50-fold, 100-fold, 120-fold, 200-fold, 250-fold, 300-fold, or 500-fold.

In aspects, VIB551 treats MG by reducing fatigue. In aspects, fatigue is measured using the Neuro-QoL Fatigue score. In aspects, treatment with VIB551 results in an improvement in the Neuro-QoL Fatigue score of at least about 1 point, about least about 2 points, at least about 3 points, at least about 4 points, at least about 5 points, at least about 6 points, at least about 7 points, at least about 8 points, at least about 9 points, at least about 10 points or more.

The V1B551 may also be used in a method of treating a subject in need of treatment for MG in which the VIB551 is administered at a dose that (i) depletes at least 90% of circulating CD20+ B cells for at least six months, and (ii) does not increase risk of infections in the subject. The dose that depletes the at least 90% of circulating CD20+ B cells for at least six months may also deplete peripheral blood CD20⁻ plasmablasts and plasma cells. The dose that depletes the at least 90% of circulating CD20+ B cells may deplete the circulating CD20+ B cells for longer than six months. It may deplete the at least 90% of circulating CD20+ B cells for at least 9 months or at least 1 year.

The dose of VIB551 that depletes at least 90% of circulating CD20+ B cells for at least six months in the method of treating also does not increase risk of infections in the MG subject. The risk of infection may not be increased in the MG subject relative to his or her risk of infections prior to the administration of VIB551. The risk of infection may not be increased in the MG subject in comparison to a subject not treated with VIB551. In aspects, the risk of infection may be a risk of infection by or resulting in typical pneumonia, beta hemolytic streptococcal infection, bronchitis, conjunctivitis, viral conjunctivitis, fungal skin infection, gastroenteritis viral, gastrointestinal infection, gingivitis, cystitis, herpes zoster, influenza, laryngitis, meningitis viral, muscle abscess, oral herpes, otitis externa, periodontitis, pneumonia, rhinitis, retinitis, pyelocystitis, retinitis, sinusitis, urinary tract infection, tinea cururis, septic shock, or upper respiratory tract infection.

The dose of VIB551 that may be used in a method of treating a subject in need of treatment for MG, wherein the VIB551 dose depletes at least 90% of circulating CD20+ B cells for at least six months, and does not increase risk of infections in the subject, may be a dose of approximately 300 mg. The approximately 300 mg may be a dose of 250 mg to 350 mg, it may be a dose of 275 to 325 mg, it may be a dose of 290 to 310 mg, it may be a dose of 205 to 305 mg, or it may be a dose of 300 mg.

In aspects, VIB551 treats MG results in the change in the Myasthenia Gravis Quality of Life-15, revised score (MGQOL-15r). In aspects, treatment with VIB551 results in an improvement in the MGQOL-15r score of at least about 1 point, about least about 2 points, at least about 3 points, at least about 4 points, at least about 5 points, at least about 6 points, at least about 7 points, at least about 8 points, at least about 9 points, at least about 10 points or more.

In aspects, VIB551 treats MG resulting in a change in the Quantitative Myasthenia Gravis (QMG) score. The QMG is a validated outcome comprised of 13 items: ocular (2 items), facial (1 item), bulbar (2 items), gross motor (6 items), axial (1 item), and respiratory (1 item). Each item has a possible score of between 0 and 3 points. The total score range is 0-39 points, with a higher score indicating more severe disease. A 3-point improvement in the QMG score is considered clinically meaningful (Barohn R, McIntire D, Herbelin L, Wolfe G, Nations S, Bryan W. Reliability testing of the quantitative myasthenia gravis score. Ann N Y Acad Sci 1998; 841:769-72; Zinman L, Baryshnik D, Bril V. Surrogate therapeutic outcome measures in subjects with myasthenia gravis. Muscle Nerve 2008; 37:172-6). In aspects, treatment with VIB551 results in a change in the QMG score of at least about 1 point, about least about 2 points, at least about 3 points, at least about 4 points, at least about 5 points, at least about 6 points, at least about 7 points, at least about 8 points, at least about 9 points, at least about 10 points or more.

In aspects, VIB551 treats MG resulting in an improvement in the Myasthenia Gravis Activities of Daily Living (MG-ADL) score. The MG ADL is a validated measure that requires no equipment and that can be administered in 10 minutes (Wolfe G I, Barohn R J, Foster B M, Jackson C E, Kissel J T, Day J W, et al; Myasthenia Gravis-IVIG Study Group. Randomized, controlled trial of intravenous immunoglobulin in myasthenia gravis. Muscle Nerve 2002; 26:549-52). In aspects, treatment with VIB551 results in an improvement in the MG-ADL at least about 1 point, about least about 2 points, at least about 3 points, at least about 4 points, at least about 5 points, at least about 6 points, at least about 7 points, at least about 8 points, at least about 9 points, at least about 10 points or higher. In aspects, treatment with VIB551 results in an improvement in the MG-ADL of at least 3 points.

In aspects, VIB551 treats MG resulting in an improvement in the Myasthenia Gravis Composite score (MGC). The MGC consists of test items from the MG-ADL and the QMG that measure symptoms and signs of MG, with weighted response options (Burns T M, Conaway M, Sanders D B; MG Composite and MG-QOL15 Study Group. The MG Composite: a valid and reliable outcome measure for myasthenia gravis. Neurology 2010; 74:1434-40). In aspects, treatment with VIB551 results in an improvement in the MGC score of at least about 1 point, about least about 2 points, at least about 3 points, at least about 4 points, at least about 5 points, at least about 6 points, at least about 7 points, at least about 8 points, at least about 9 points, at least about 10 points or more.

The dose of VIB551 that may be used in a method of treating a subject in need of treatment for MG, may be a dose administered intravenously at an interval of approximately once every 6 months, or once every 7 months, or once every 8 months, or once every 9 months, or once every 10 months, or once every 11 months, or once a year. In aspects, VIB551 administered in the methods disclosed herein may be at an interval of approximately every 6 months. The approximately every 6 months may be administration every 6 months, every 180 days, every between 170 and 190 days, every between 175 and 185 days, every between 175 and 190 days, or every between 170 and 185 days. The approximately every 6 months may be administration every 26 weeks, every 25 weeks, every 27 weeks, every between 25 and 27 weeks, every between 25 and 26 weeks, or every between 26 and 27 weeks. Prior to the administering the VIB551 every approximately 6 months in the methods disclosed herein, an initial VIB551 dose may be administered to the MG subject. The initial VIB551 dose may be administered approximately 2 weeks before the approximately every 6-month VIB551 dosing. The administering the initial VIB551 dose approximately 2 weeks before the approximately every 6-month VIB551 dosing may be the administering of the initial VIB551 dose 12 days, 13 days, 14 days, 15 days, or 16 days before the approximately 6 months VIB551 dosing. The initial VIB551 dose may or may not be co-administered with oral corticosteroids or any standard of treatment dose. In aspects, VIB551 is administered intravenously.

In aspects, the disclosure is directed to a method of treating myasthenia gravis (MG), the method comprising: administering VIB551 to a subject in need of treatment for MG, wherein the VIB551 is administered intravenously at a dose of 300 mg every 6 months. In aspects, two weeks prior to the administering the 300 mg VIB551 every 6 months, an initial 300 mg VIB551 dose is administered to the subject. In aspects, the disclosure is directed to a method of treating a subject diagnosed with MG, the method comprising: administering VIB551 to a subject in need of treatment for MG, wherein the VIB551 is administered intravenously with a first initial dose of 300 mg, a second initial dose of 300 mg two weeks after the first initial dose, and subsequent doses of 300 mg every 6 months following the first initial dose.

In aspects, the VIB551 may be administered at a dose of about 300 mg. In aspects, the VIB551 may be administered at a dose of about 250 mg to about 350 mg, about 275 mg to about 325 mg, about 290 mg to about 310 mg, about 205 mg to about 305 mg, or it may be a dose of 300 mg. In aspects, the subject may receive one or more initial doses of V1B551. In aspects, the subject may receive, one, two, three or more initial doses. In aspects, the initial dose may be about 300 mg. In aspects, the VIB551 may be administered at an initial dose of about 250 mg to about 350 mg, about 275 mg to about 325 mg, about 290 mg to about 310 mg, about 205 mg to about 305 mg, or an initial dose of 300 mg. In aspects, VIB551 may be administered intravenously with a first initial dose of about 300 mg, a second initial dose of about 300 mg two weeks after the first initial dose, and subsequent doses of about 300 mg every 6 months following the first initial dose.

### Additional Therapies

In aspects, VIB551 is administered with one or more additional therapies. In aspects, the one or more additional therapies are one or more standard of care therapies. In aspects, an additional therapy is selected from the group consisting of: immunosuppressant, anticholinesterase, surgery (e.g., thymectomy), plasmapheresis, and combinations thereof.

In aspects, an additional therapy comprises an immunosuppressant. Immunosuppressants help reduce or eliminate antibodies that cause MG weakness. At the same time, they may also reduce the body's production of antibodies-which may make a treated subject susceptible to infection and other diseases.

In aspects, the additional therapy is a corticosteroid. In aspects, the corticosteroid is prednisone. While prednisone can be very effective in treating myasthenia, it carries the risk of serious side effects; their severity depends on the dosage and length of time prednisone is used. These include insomnia, mood changes, weight gain, fluid retention, reduced resistance to infection, increased susceptibility to diabetes, high blood pressure, osteoporosis, glaucoma, cataracts, and stomach ulcers, plus a host of other less common side effects. Accordingly, in aspects, a subject experiences a reduction in prednisone-related side effects with administered Inebilizumab.

In aspects, the one or more additional therapies are one or more of azathioprine, mycophenolate mofetil, mycophenolic acid, or tacrolimus. In aspects, a dosage of the one or more of azathioprine, mycophenolate mofetil, mycophenolic acid, or tacrolimus is at most about: azathioprine≤3 mg/kg/day, mycophenolate mofetil≤3 g/day, mycophenolic acid≤1440 mg/day, and/or tacrolimus≤3 mg/day.

In aspects, the additional therapy comprises an anticholinesterase. Although an anticholinesterase medication does not directly counteract the abnormal immune system attack in MG, they may partially or completely control MG symptoms in some subjects. In aspects, an anticholinesterase comprises pyridostigmine bromide.

In aspects, an additional therapy comprises an anti-CD20 antibody that is not rituximab. In aspects, subjects that have chronic MG are not administered rituximab as rituximab primarily targets short-lived CD20-positive plasma cell precursors and/or reducing memory cells. By contrast, in chronic MG, a subject may retain a pool of CD20-negative plasma cells that contributes to disease pathology and are not depleted by rituximab. Accordingly, Inebilizumab may produce longer-lasting B-cell depletion than anti-CD20 agents (e.g., rituximab) by potentially targeting a greater proportion of B cells that are driving immunopathogenesis, including some mature plasma cells. In aspects, administration of Inebilizumab results in longer-lasting B-cell depletion as compared to an anti-CD20 agent. In aspects, a subject administered Inebilizumab experiences B-cell depletion for at least about or at most about: 1 day, at least 2 days, at least 3 days, at least 4 days, at least 5 days at least 6 days, at least 7 days, at least 8 days, at least 9 days, at least 10 days, at least 15 days, at least 20 days, at least 25 days, or at least 30 days. In aspects, B cell depletion may persist for at least 1 week, at least 2 weeks, at least 3 weeks, at least 4 weeks, at least 5 weeks, at least 6 weeks, at least 7 weeks, at least 8 weeks, at least 9 weeks, or at least 10 weeks. In aspects, B cell depletion may persist for at least 1 month, at least 2 months, at least 3 months, at least 4 months, at least 5 months, at least 6 months, at least 7 months, at least 8 months, at least 9 months, at least 10 months, at least 11 months or 12 months.

### Assessments

Quality of Life in Neurological Disorders Fatigue score

In aspects, a method provided herein comprises determining a Quality of Life in Neurological Disorders Fatigue score. In aspects, post treatment with Inebilizumab, the Quality of Life in Neurological Disorders Fatigue score is reduced as compared to an otherwise comparable subject not administered Inebilizumab. In aspects, a score is reduced by at least about 0-2, 1-3, 2-5, or 3-5 points post treatment with Inebilizumab. In aspects, a score is reduced by at least about 1, 2, 3, 4, 5, or 6 points post treatment with Inebilizumab.

### Patient Global Impression of Change (PGIC)

In aspects, a method provided herein comprises determining a level of Patient Global Impression of Change (PGIC). The self-report measure PGIC reflects a subject's belief about the efficacy of treatment. PGIC is a 7-point scale depicting a subject's rating of overall improvement. In aspects, post treatment with Inebilizumab, the PGIC is reduced as compared to an otherwise comparable subject not administered Inebilizumab. In aspects, a score is reduced by at least about 0-2, 1-3, 2-5, or 3-5 points post treatment with Inebilizumab. In aspects, a score is reduced by at least about 1, 2, 3, 4, 5, or 6 points post treatment with Inebilizumab.

### MG Composite (MGC) Total Score

In aspects, a method provided herein comprises determining a MG composite (MGC) total score. The Myasthenia Gravis Composite score has been validated as an outcome measure of signs and symptoms for subjects with myasthenia gravis. The score was first described by Burns et al. (2008) and has been replicated for the use in MGC in the Criteria from Burns et al. (2010). In aspects, post treatment with Inebilizumab, the MGC score is reduced as compared to an otherwise comparable subject not administered Inebilizumab. In aspects, a score is reduced by at least about 0-2, 0-5, 5-10, 10-15, 20-30, or 25-35 points post treatment with Inebilizumab. In aspects, a score is reduced by at least about 1, 2, 3, 4, 5, 10, 15, 20, 30, or 40 points post treatment with Inebilizumab.

### Biologic

In aspects, a method provided herein can comprise a biologic assessment. In aspects, an assessment comprises determining anti-AChR and anti-MuSK antibody titers. In aspects, the antibody titers can be correlated with clinical outcomes post treatment with Inebilizumab. Antibody titers can be ascertained by way of a blood test to evaluate serum antibodies.

In aspects, an assessment comprises B-cell subset phenotyping. In aspects, a method comprises determining the presence or level of one or more B cells selected from the group consisting of: transitional, naive, plasma, and memory B-cells. In aspects, a method comprises identifying B-cells by way of CD20, CD27, and CD19 in a subject serum sample. A method provided herein can be effective in reducing or eliminating one or more B-cell subtypes. In aspects, post administration, a level of B-cells in a subject in reduced by at least about 5%, 10%, 20%, 40%, 60%, 80%, or 100%. In aspects, post administration, a level of B-cells in a subject in reduced by at least about 1-fold, 5-fold, 10-fold, 40-fold, 60-fold, 80-fold, 100-fold, 200-fold, 500-fold, or 1000-fold.

In aspects, an assessment comprises determining B-cell receptor repertoire. Each B cell expresses a single B cell receptor (BCR), and the diverse range of BCRs expressed by the total B cell population of a subject is termed the ‘BCR repertoire’ herein. In aspects, a BCR repertoire is determined before, concurrent, or after treatment with Inebilizumab. BCR repertoire can be evaluated by sequencing.

In aspects, an assessment comprises gene expression profiling of immune cells (e.g., B-cells). Gene expression profiling can be the determination of the pattern of genes expressed, at the level of transcription, under specific circumstances or in a specific cell, for example B-cell, to give a global picture of cellular function. Gene expression profiling measures mRNA levels, showing the pattern of genes expressed by a cell at the transcription level. In aspects, a method comprises measuring relative mRNA amounts in two or more experimental conditions (e.g., placebo or Inebilizumab and/or AChR-Ab+ or MuSK-Ab+, then assessing which conditions resulted in specific genes being expressed.

Those skilled in the art will recognize or be able to ascertain using no more than routine experimentation, many equivalents to the aspects described herein. Such equivalents are intended to be encompassed by the appended claims.

### Examples of Non-Limiting Embodiments of the Disclosure

Embodiments of the present subject matter disclosed herein may be beneficial alone or in combination with one or more other embodiments. Without limiting the foregoing description, certain non-limiting embodiments of the disclosure are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

### Embodiments

### Embodiment Set 1

1. A method of treating myasthenia gravis (MG), the method comprising: administering Inebilizumab to a subject in need of treatment for MG, wherein the Inebilizumab is administered intravenously at a dose of about 300 mg every 6 months.

2. The method of embodiment 1, wherein two weeks prior to administering the 300 mg Inebilizumab every 6 months, an initial 300 mg Inebilizumab dose is administered to the subject.

3. A method of treating a subject diagnosed with MG, the method comprising: administering Inebilizumab to a subject diagnosed with MG, wherein the Inebilizumab is administered intravenously at a first dose of 300 mg, a second dose of 300 mg two weeks after the first dose, and subsequent doses of 300 mg every 6 months following the first dose.

4. A method of treating a subject diagnosed with MG, the method comprising: administering Inebilizumab to a subject diagnosed with MG, wherein the Inebilizumab is administered at a dose that: (i) depletes at least 90% of circulating CD20+ B cells for at least six months; and (ii) does not increase incidence of an infection in the subject.

5. The method of embodiment 4, wherein the Inebilizumab further depletes peripheral blood CD20⁻ plasmablasts and plasma cells within about 8 days following the administering.

6. The method of embodiment 4 or embodiment 5, wherein the dose is 300 mg.

7. A method of reducing MG-related disability, the method comprising: administering Inebilizumab to a subject in need of treatment for MG, wherein the Inebilizumab is administered intravenously at a first dose of 300 mg, a second dose of 300 mg two weeks after the first dose, and subsequent doses of 300 mg every 6 months following the first initial dose.

8. A method of reducing the frequency of MG exacerbations, the method comprising: administering Inebilizumab to a subject in need of treatment for MG, wherein the Inebilizumab is administered intravenously with at a first dose of 300 mg, a second dose of 300 mg two weeks after the first dose, and subsequent doses of 300 mg every 6 months following the first dose.

9. The method of any one of the preceding embodiments, wherein the subject is acetylcholine receptor antibody positive (AChR-Ab+).

10. The method of any one of the preceding embodiments, wherein the subject is muscle-specific kinase-antibody positive (MuSK-Ab+).

11. The method of any one of the preceding embodiments, wherein the subject is acetylcholine receptor antibody positive (AChR-Ab+) and muscle-specific kinase-antibody positive (MuSK-Ab+).

12. The method of any one of the preceding embodiments, wherein administration of the Inebilizumab reduces the frequency of MG exacerbations.

13. The method of any one of the preceding embodiments, wherein administration of the Inebilizumab reduces fatigue as determined by Neuro-QoL Fatigue score.

14. The method of any one of the preceding embodiments, wherein the subject is further administered one or more additional therapies.

15. The method of embodiment 14, wherein the one or more additional therapies comprise one or more standard-of-care therapies.

16. The method of embodiment 15, wherein the standard-of-care therapy comprises a corticosteroid, a nonsteroidal immunosuppressive therapy, or both.

17. The method of embodiment 16, comprising the corticosteroid, wherein the corticosteroid is prednisone.

18. The method of embodiment 14, wherein the one or more additional therapies are one or more of azathioprine, mycophenolate mofetil, mycophenolic acid, or tacrolimus.

19. The method of any one of the preceding embodiments, wherein the Inebilizumab comprises a heavy chain variable region (VH) comprising the amino acid of SEQ ID NO:1, and a light chain variable region (VL) comprising the amino acid of SEQ ID NO: 2.

20. The method of any one of embodiments 4-6, wherein the Inebilizumab is administered intravenously.

21. The method of any one of the preceding embodiments, wherein the administration of the Inebilizumab treats the MG as determined by: a) a reduction in MG activities of daily living score; b) a reduction of a MG score; c) an increase in a quality-of-life score; and/or d) a reduction in incidence of exacerbations.

22. The method of any one of the preceding embodiments, wherein the MG is refractory MG.

23. The method of any one of the preceding embodiments, wherein the subject in need of treatment for MG has uncontrolled MG, on a standard-of-care therapy, as determined by one or more of: a) a MG Foundation of America classification of II, III, or IV; b) an MG activities of daily living (MG-ADL) score≥6, with >50% of this score being attributed to non-ocular items; or c) a quantitative MG (QMG) score≥11.

24. The method of embodiment 17, wherein if the subject in need of treatment for MG is receiving a dosage of prednisone over 5 mg/day or equivalent, the subject will undergo tapering of the prednisone dosage to 5 mg/day.

25. The method of embodiment 18, comprising a maximum dosage of: a) tacrolimus≤3 mg/day; b) azathioprine≤3 mg/kg/day; c) mycophenolate mofetil≤3 g/day; and/or d) mycophenolic acid≤1440 mg/day.

26. The method of embodiment 23, comprising b), wherein the MG-ADK score is reduced after the administration of Inebilizumab.

27. The method of embodiment 26, wherein the reduction is of at least about 2 points.

28. The method of any one of the preceding embodiments, comprising determining: a) B-cell subset phenotype; b) B-cell receptor repertoire; c) B-cell gene expression profiling; or d) any combination thereof.

29. The method of any one of the preceding embodiments, wherein the administering is effective in reducing or eliminating mature plasma cells in the subject.

30. The method of any one of the preceding embodiments, wherein the administration results in longer-lasting B-cell reduction or elimination as compared to an otherwise comparable method comprising administration of an anti-CD20 therapy.

### Embodiment Set 2

1. A method of treating myasthenia gravis (MG), the method comprising administering to a subject in need of treatment for MG an antibody that comprises a heavy chain variable region (VH) comprising the amino acid of SEQ ID NO: 1, and a light chain variable region (VL) comprising the amino acid of SEQ ID NO: 2, wherein the antibody is administered intravenously at a dose of from about 250 mg to about 350 mg every 6 months, thereby treating the MG.

2. The method of embodiment 1, wherein two weeks prior to the administering, an initial dose of the antibody is administered to the subject, and wherein the initial dose is from about 250 mg to about 350 mg.

3. The method of any one of embodiments 1-2, wherein a second dose of the antibody of about 300 mg is administered two weeks after the administering.

4. The method of any one of the preceding embodiments, wherein the administering is effective in (i) depleting at least about 90% of circulating CD20+ B cells for at least six months; (ii) does not increase incidence of an infection in the subject; or (iii) (i) and (ii).

5. The method of embodiment 4, wherein the administering reduces a level of peripheral blood CD20⁻ plasmablasts and plasma cells within about 8 days following the administering.

6. The method of embodiment 4 or embodiment 5, wherein the dose is about 300 mg.

7. The method of any one of the preceding embodiments, wherein the administering is effective in reducing MG-related disability.

8. The method of any one of the preceding embodiments, wherein the administering is effective in reducing frequency of MG exacerbations.

9. The method of any one of the preceding embodiments, wherein the subject is acetylcholine receptor antibody positive (AChR-Ab+).

10. The method of any one of the preceding embodiments, wherein the subject is muscle-specific kinase-antibody positive (MuSK-Ab+).

11. The method of any one of the preceding embodiments, wherein the subject is acetylcholine receptor antibody positive (AChR-Ab+) and muscle-specific kinase-antibody positive (MuSK-Ab+).

12. The method of embodiment 8, wherein the frequency of MG exacerbations is reduced by at least about 1-fold as compared to an otherwise comparable subject lacking the administering.

13. The method of any one of the preceding embodiments, wherein the administering is effective in reducing fatigue in the subject as determined by Neuro-QoL Fatigue score.

14. The method of any one of the preceding embodiments, wherein the subject is further administered one or more additional therapies.

15. The method of embodiment 14, wherein the one or more additional therapies comprise one or more standard-of-care therapies.

16. The method of embodiment 15, wherein the one or more standard-of-care therapies comprises a corticosteroid, a nonsteroidal immunosuppressive therapy, or both.

17. The method of embodiment 16, wherein the one or more standard-of-care therapies comprises the corticosteroid, and wherein the corticosteroid comprises prednisone.

18. The method of embodiment 14, wherein the one or more additional therapies are one or more of azathioprine, mycophenolate mofetil, mycophenolic acid, or tacrolimus.

19. The method of any one of the preceding embodiments, wherein the antibody is Inebilizumab.

20. The method of any one of the preceding embodiments, wherein the antibody is administered at a dose of about 300 mg.

21. The method of any one of the preceding embodiments, wherein the administering treats the MG as determined by: a) a reduction in MG activities of daily living score; b) a reduction of a MG score; c) an increase in a quality-of-life score; and/or d) a reduction in incidence of exacerbations.

22. The method of any one of the preceding embodiments, wherein the MG is refractory MG.

23. The method of any one of the preceding embodiments, wherein the subject in need of treatment for MG has uncontrolled MG, on a standard-of-care therapy, as determined by one or more of: a) a MG Foundation of America classification of II, III, or IV; b) an MG activities of daily living (MG-ADL) score≥6, with >50% of this score being attributed to non-ocular items; or c) a quantitative MG (QMG) score≥11.

24. The method of embodiment 17, wherein if the subject in need of treatment for MG is receiving a dosage of prednisone over 5 mg/day or equivalent, the subject will undergo tapering of the prednisone dosage to 5 mg/day.

25. The method of embodiment 18, comprising a maximum dosage of: a) tacrolimus≤3 mg/day; b) azathioprine≤3 mg/kg/day; c) mycophenolate mofetil≤3 g/day; and/or d) mycophenolic acid≤1440 mg/day.

26. The method of embodiment 23, wherein the subject in need of treatment for MG has uncontrolled MG, on a standard-of-care therapy, as determined by the MG-ADL score of ≥6, with >50% of the score being attributed to non-ocular items, and wherein the MG-ADK score is reduced after the administering.

27. The method of embodiment 26, wherein the reduction is of at least about 2 points.

28. The method of any one of the preceding embodiments, comprising determining: a) a B-cell subset phenotype; b) a B-cell receptor repertoire; c) a B-cell gene expression profiling; or d) any combination thereof.

29. The method of any one of the preceding embodiments, wherein the administering is effective in reducing or eliminating mature plasma cells in the subject.

30. The method of any one of the preceding embodiments, wherein the administering results in longer-lasting B-cell reduction or elimination as compared to an otherwise comparable method comprising administration of an anti-CD20 therapy.

31. A method of treating myasthenia gravis (MG), the method comprising: (a) administering a standard-of-care therapy in an amount sufficient to treat MG in a subject in need thereof; and (b) administering a pharmaceutical composition that comprises an antibody that comprises a heavy chain variable region (VH) comprising the amino acid of SEQ ID NO:1, and a light chain variable region (VL) comprising the amino acid of SEQ ID NO: 2 at a dose of from about 250 mg to about 350 mg.

32. The method of embodiment 31, wherein the standard-of-care therapy comprises a corticosteroid.

33. The method of embodiment 31, wherein the standard-of-care therapy comprises a nonsteroidal immunosuppressive therapy.

34. The method of embodiment 31, wherein the standard-of-care therapy comprises a corticosteroid and a nonsteroidal immunosuppressive therapy.

35. The method of embodiment 32, wherein the corticosteroid comprises prednisone.

36. The method of embodiment 31, wherein the antibody is Inebilizumab.

37. The method of embodiment 31, wherein the dose is from about 275 mg to about 325 mg, from about 290 mg to about 310 mg, or 205 mg to about 305 mg.

38. The method of embodiment 37, wherein the dose is about 300 mg.

39. The method of any preceding embodiments, wherein the subject has a CD19+ B-cell count≥40 cells/μL prior to administration of the antibody.

40. The method of any preceding embodiments, wherein the subject has a myasthenia gravis foundation of America (MGFA) clinical classification selected from class II, class III, or class IV.

41. The method of any preceding embodiments, wherein the subject has anti-AChR antibodies or anti-MuSK antibodies, or both.

42. The method of any preceding embodiments, wherein the subject does not have an immunodeficiency disorder.

### EXAMPLES

### Example 1—A Randomized, Double-blind, Multicenter, Placebo-controlled Phase 3 Study with Open-label Period to Evaluate the Efficacy and Safety of Inebilizumab in Adults with Myasthenia Gravis

This study is a phase 3, randomized, double-blind, placebo-controlled study. The target population is adult subjects aged≥18 years with acetylcholine receptor antibody positive (AChR-Ab+) or muscle-specific kinase-antibody positive (MuSK-Ab+) generalized MG. Approximately 270 adult subjects (188 AChR-Ab+ and 82 MuSK-Ab+) are enrolled.

Primary objective: To assess whether Inebilizumab can reduce MG-related disability.

Four secondary objectives: 1. To evaluate whether Inebilizumab can reduce the frequency of MG exacerbations. 2. To evaluate whether Inebilizumab can improve MG-related quality of life. 3. To evaluate the safety and tolerability of Inebilizumab in MG. 4. To characterize the pharmacokinetic (PK) profile and immunogenicity of Inebilizumab in subjects with MG.

Exploratory objectives: 1. To evaluate whether Inebilizumab can reduce fatigue in MG. 2. To evaluate the ability of Inebilizumab to elicit minimal symptom expression. 3. To evaluate the effect of Inebilizumab on corticosteroid usage. 4. To evaluate whether Inebilizumab can reduce healthcare resource utilization. 5. To evaluate the pharmacodynamic (PD) profile of Inebilizumab in MG. 6. To evaluate the effect of Inebilizumab on disease biomarkers.

Primary endpoint: Change from baseline in Myasthenia Gravis Activities of Daily Living (MG ADL) score at end of the RCP (Week 52 for AChR Ab+ population and Week 26 for MuSK-Ab+ population).

Key secondary endpoints: 1. Change in Quantitative Myasthenia Gravis (QMG) scores at the end of the RCP. 2. Proportion of subjects with both (1)≥3-point improvement in MG-ADL at end of RCP and (2) no use of rescue therapy during the RCP. 3. Change in MG-ADL at Week 26 in the AChR-Ab+ population. As a secondary outcome, the proportion of patients with a≥3-point improvement in MG-ADL at the end of the RCP without the need for rescue therapy is compared between active and placebo groups.

Additional secondary endpoints: 4. Time to first exacerbation. 5. Change in Myasthenia Gravis Composite (MGC) score at the end of the RCP. 6. Change in Myasthenia Gravis Quality of Life-15, revised (MGQOL-15r) score at the end of the RCP. 7. Change in Subject Global Impression of Change score at the end of the RCP. 8. The safety and tolerability of Inebilizumab as measured by the incidence of treatment-emergent adverse events (TEAEs), adverse events of special interest (AESIs), and treatment-emergent serious adverse events (TESAEs). Laboratory measurements are evaluated. 9. PK profile of Inebilizumab. 10. Anti-drug antibody (ADA) status and titer.

Exploratory endpoints: 1. Change in Quality of Life in Neurological Disorders (Neuro-QOL) Fatigue score at the end of the RCP. 2. Proportion achieving minimal symptom expression, defined as MG-ADL=0 or 1, at the end of the RCP. 3. Myasthenia Gravis Foundation of America Post-Intervention Status at the end of the RCP. 4. Corticosteroid usage. 5. Healthcare resource utilization. 6. PD profile, as measured by CD20+ B-cell count. 7. Change in anti-acetylcholine receptor (anti-AChR) and anti-muscle-specific kinase (anti-MuSK) antibody titers and correlation with clinical outcomes (not done in China) 8. B-cell subset flow cytometry. 9. B-cell repertoire profiling (sub study in selected countries).

Subjects with MG who are positive for anti-AChR or anti-MuSK antibodies are enrolled and analyzed separately as 2 populations: (1) AChR-Ab+ and (2) MuSK-Ab+. Subjects who do not have anti-AChR or anti-MuSK antibodies are not enrolled. Subjects with MGFA classification II, III, or IV disease, MG ADL score≥6, QMG score>11, and use of a corticosteroid and/or non-steroidal immunosuppressant are included in the study. These criteria define a population of subjects with generalized MG and inadequate symptom control.

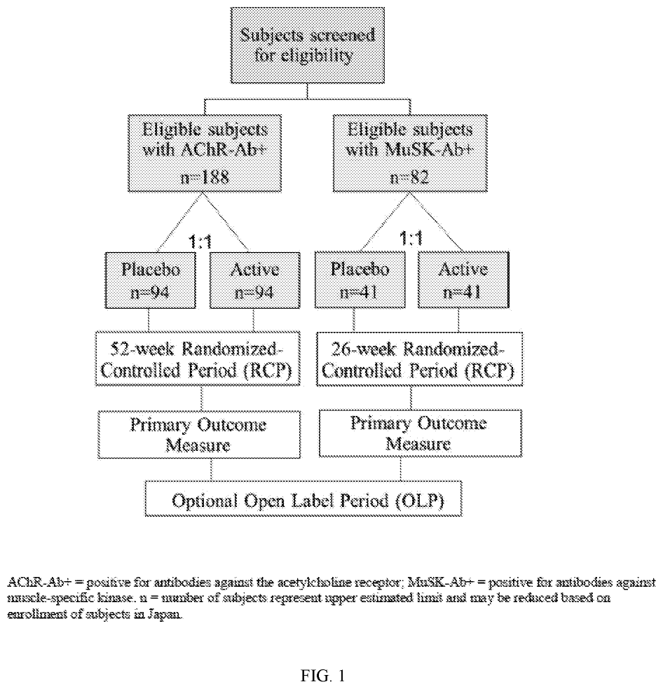

Within each population, subjects are stratified by region first (non-Japan vs Japan). In the non-Japan population, subjects are further stratified according to baseline disease severity (‘Baseline QMG score=11-15’ vs ‘Baseline QMG score≥16’) and baseline corticosteroid use (‘prednisone>5 mg/day’ vs ‘prednisone≤5 mg/day’) and randomized 1:1 to receive either IV Inebilizumab or placebo within each stratum (FIG. 1). To quantify disease severity for stratification purposes, the QMG was chosen over the MG-ADL because the results are more likely to be consistent between individuals since the QMG is based on objective examination and the MG-ADL is based on subjective reporting.

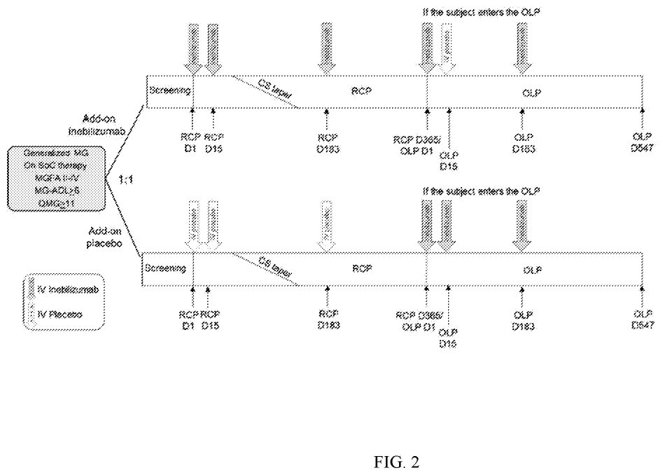

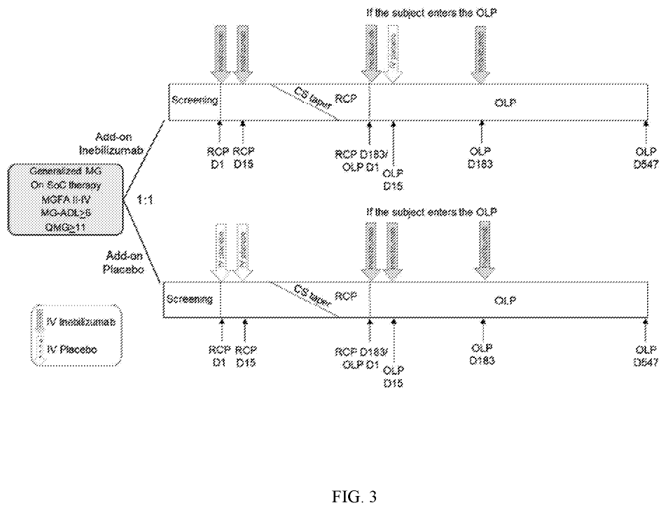

In the Japan population, no further stratification is applied due to the small sample size, and subjects are randomized 1:1 to receive either IV Inebilizumab or placebo. Subjects are allowed to enter the study on a standard of care regimen, including acetylcholinesterase inhibitors (AChEI), corticosteroids, and/or azathioprine, mycophenolate mofetil, and mycophenolic acid. In Japan, subjects are allowed to enter the study on tacrolimus. The duration of the RCP is 52 weeks for the AChR-Ab+ population (FIG. 2) and 26 weeks for the MuSK-Ab+ population (FIG. 3); the different timepoint is due to expected differences in the kinetics of the treatment response.

Subjects who enter the study on prednisone>5 mg/day (or equivalent other corticosteroid) have their corticosteroid dose tapered per protocol, starting at Week 4 of the RCP. Steroid tapering continues until the subject is on a dose of prednisone 5 mg/day; the prednisone 5 mg/day dose is continued until the end of the RCP. Corticosteroids are not initiated if the subject is not being treated with them at the time of randomization.

The dose of azathioprine, mycophenolate mofetil, mycophenolic acid, and AChEI remains stable throughout the RCP. For subjects on tacrolimus (allowed in Japan only), the dose should not be increased during the RCP but may be reduced, based on the judgement of the investigator, for safety reasons.

All subjects who complete the RCP have the option to enroll in a 1.5-year OLP. In the OLP, further tapering of azathioprine, mycophenolate mofetil, mycophenolic acid, tacrolimus (if applicable) and corticosteroids is recommended.

### Dose and Treatment Regimen Rationale

In this study, the dosing regimen of 300 mg IV on Day 1 and Day 15, followed by a single 300 mg infusion approximately every 6 months thereafter is tested for safety and efficacy of Inebilizumab in MG. See FIG. 2 and FIG. 3.
Treatment group 1: AChR-Ab+ population (active)—Inebilizumab 300 mg administered intravenously (IV) on Days 1, 15, and 183 of the RCP.
Treatment group 2: AChR-Ab+ population (placebo)—IV placebo on Days 1, 15, and 183 of the RCP.
Treatment group 3: MuSK-Ab+ population (active)—Inebilizumab 300 mg IV on Days 1 and 15 of the 26-week RCP.
Treatment group 4: MuSK-Ab+ population (placebo)—IV placebo on Days 1 and 15 of the 26-week RCP.

Following the RCP, subjects from an active treatment group who elect to enter the OLP receive Inebilizumab 300 mg IV on OLP Day 1, IV placebo on OLP Day 15 (to avoid potential unblinding), and Inebilizumab 300 mg IV on OLP Day 183. Following the RCP, subjects from a placebo treatment group who elect to enter the OLP receive Inebilizumab 300 mg IV on OLP Days 1, 15, and 183.

### Rationale for Study Population

The study enrolls male and female subjects≥18 years old, with AChR-Ab+ or MuSK-Ab+ generalized MG. The AChR-Ab+ and MuSK-Ab+ populations are analyzed separately due to different immunopathogenic features. As there are also distinct differences between the clinical manifestations and responsiveness to treatment, these populations may respond differently to Inebilizumab and as such, are best studied independently.

The inclusion criteria are designed to enroll subjects with sufficient disease activity to merit the use of a biologic disease-modifying drug. Only subjects who are MGFA class II (mild weakness affecting other than ocular muscles), III (moderate weakness affecting other than ocular muscles), or IV (severe weakness affecting other than ocular muscles) are eligible for the study. Subjects with MGFA classifications below II have ocular signs and symptoms only and their disease is too mild for inclusion. Subjects with MGFA classifications above IV require mechanical ventilation (Jaretzki et al, 2000) and their disease is too severe for inclusion.

The study enrolls subjects with generalized MG who are receiving selected standard of care therapies. Subjects receiving corticosteroids at the time of randomization stay at the same corticosteroid dose, and dose tapering begins at Week 4 of the RCP. The maximum allowed dose of prednisone at the time of randomization is 40 mg/day or 80 mg every other day. The corticosteroid dose must not have been increased within the 4 weeks prior to randomization; reductions in dose during the screening period are allowed.

Subjects entering the study on an allowed non-steroidal IST must have been on the drug continuously for ≥6 months with no dose increase in the 4 months prior to randomization. Subjects remain on the same dose of non-steroidal IST for the duration of the RCP unless dose reduction is deemed necessary for safety reasons. Azathioprine, mycophenolate mofetil, and mycophenolic acid are allowed non-steroidal ISTs. Tacrolimus is allowed in Japan only since it is the standard of care for treatment of MG in Japan.

Subjects on a stable dose of pyridostigmine≤480 mg/day are allowed to enroll. The pyridostigmine dose must have been stable for at least 2 weeks prior to randomization. The pyridostigmine dose must remain stable throughout the RCP unless dose reduction is deemed necessary for safety reasons. No increase in pyridostigmine is allowed in the study. Pyridostigmine should be held for at least 12 hours prior to every study visit so that objective testing can be performed without the confounding effect of pyridostigmine

### Inclusion Criteria

To be included in this study, each subject must satisfy all the following criteria:
1. Male or female subjects≥18 years old.
2. Written informed consent and any locally required authorization (e.g., Health Insurance Portability and Accountability Act [HIPAA] in the United States of America, European Union Data Privacy Directive in the European Union) obtained from the subject prior to performing any protocol-related procedures, including screening evaluations. In countries where the legal adult age is >18, subjects≥18 but under legal age for that country must also provide assent and have consent of a parent or other legally authorized representative.
3. Diagnosis of MG defined as:
   a. Positive serologic test for anti-AChR or anti-MuSK antibody titers as confirmed at screening (one retest allowed), and
   b. At least one of the following:
      History of abnormal neuromuscular transmission test results demonstrated by single-fiber electromyography or repetitive nerve stimulation; or
      History of positive anticholinesterase test (e.g., edrophonium chloride test); or
      Subject demonstrated improvement in MG signs on oral cholinesterase inhibitors, as assessed by the treating physician; or
      Clinical syndrome consistent with a diagnosis of MG, and not otherwise explained by another condition.
4. MGFA Clinical Classification Class II, III, or IV at the time of screening and randomization.
5. MG-ADL score of 6 or greater at screening and at randomization with >50% of this score attributed to non-ocular items.
6. QMG score of 11 or greater at screening and at randomization.
7. Subjects must be on:
   a. Corticosteroids only, with no dose increase within 4 weeks prior to randomization, or
   b. One allowed non-steroidal IST, with continuous use for at least 6 months prior to randomization and no dose increase within 4 months prior to randomization, or
   c. Combination of (1) corticosteroids with no dose increase within 4 weeks prior to randomization and (2) one allowed non-steroidal IST with continuous use for at least 6 months prior to randomization and no dose increase within 4 months prior to randomization.
   Allowed ISTs, alone or in combination with corticosteroids, are azathioprine, mycophenolate mofetil, and mycophenolic acid.
   Tacrolimus is allowed in Japan only, at a dose of ≤3 mg/day, with continued use for at least 6 months prior to randomization and no dose increase within 4 months prior to randomization.
8. Willing and able to comply with the protocol, complete study assessments, and return for follow-up visits.
9. Females of childbearing potential who are sexually active with a non-sterilized male partner must use at least one highly effective contraception method (Error! Reference source not found.) from the time of screening and for 6 months after the final dose of IP. Periodic abstinence, the rhythm method, and the withdrawal method are not acceptable methods of contraception.

Females of childbearing potential are defined as those who are not surgically sterile (i.e., bilateral tubal ligation, bilateral oophorectomy, or complete hysterectomy) or those who are not postmenopausal (defined as 12 months with no menses without an alternative medical cause and a follicle-stimulating hormone [FSH] level in the postmenopausal range [≥16.70 mIU/mL]). If the FSH level is not in the postmenopausal range in a subject with amenorrhea, she may still enroll in the study but must follow the same contraception requirements as women of childbearing potential.
10. Non-sterilized males who are sexually active with a female partner of childbearing potential must use a condom from Day 1 for the duration of the study and for 3 months after the last dose of IP. Because male condom is not a highly effective contraception method, it is strongly recommended that female partners of a male study subject also use a highly effective method of contraception throughout this period (Error! Reference source not found.).
11. Vital signs, electrocardiogram (ECG), and laboratory parameters within the normal ranges at screening, or, if outside normal ranges, deemed not clinically significant by the Investigator.

### Exclusion Criteria

If an individual meets any of the following criteria, he or she is ineligible for this study:
1. Any condition that, in the opinion of the Investigator, would place the subject at unacceptable risk of complications, interfere with evaluation of the IP, or confound the interpretation of subject safety or study results.
2. Lactating or pregnant females, or females who intend to become pregnant anytime from signing the informed consent form (ICF) throughout the RCP plus 6 months following last dose of IP.
3. History of drug or alcohol abuse within <1 year prior to screening, or any condition associated with poor compliance as judged by the Investigator.
4. Employees of the Sponsor, contract research organization (CRO), site staff, and their family members.
5. Currently committed to an institution by way of official or judicial order.
6. Subjects diagnosed with congenital myasthenic syndromes.
7. Known immunodeficiency disorder, including human immunodeficiency virus (HIV) infection.
8. Thymectomy within ≤12 months prior to baseline (Day 1) visit or planned thymectomy during the duration of the RCP.
9. Receipt of the following medications or treatments at any time prior to randomization:
   c. Alemtuzumab (Lemtrada®, Campath®)
   d. Total lymphoid irradiation
   e. Bone marrow transplant
   f. T-cell vaccination therapy
   g. Natalizumab (Tysabri®)
10. Receipt of rituximab (MabThera®, Rituxan®), ocrelizumab (Ocrevus®), ofatumumab (Arzerra®), obinutuzumab (Gazyva®), inebilizumab, or any experimental B-cell depleting agent within the 6 months prior to Day 1, unless the subject has a CD19+ B-cell count≥40 cells/μL according to the central laboratory at screening.
11. Receipt within the 3 months prior to Day 1:
   a. Tocilizumab (Actemra®)
   b. Belimumab (Benlysta®)
   c. Eculizumab (Soliris®)
   d. Cyclophosphamide (Cytoxan®)
12. Receipt within the 4 weeks prior to Day 1:
   a. Cyclosporine (except eye drops)
   b. Tacrolimus (except topical) (tacrolimus≤3 mg/day is allowed in Japan only)
   c. Methotrexate
   d. Intravenous immunoglobulin (IVIg)
   e. Plasma exchange (PLEX) treatment
13. Current use of:
   a. Prednisone>40 mg/day or >80 mg over a 2-day period (or equivalent dose of other corticosteroids)
   b. Pyridostigmine>480 mg/day or unstable dose in the 2 weeks prior to Day 1
   c. Azathioprine>3 mg/kg/day
   d. Mycophenolate mofetil>3 g/day or mycophenolic acid>1440 mg/day
14. Concurrent/previous enrollment in another clinical study involving an investigational treatment within 4 weeks or 5 half-lives of the investigational treatment, whichever is longer, prior to Day 1.
15. Receipt of a live attenuated vaccine within 4 weeks prior to randomization. Administration of inactivated (killed) vaccines is acceptable.
16. History of severe allergic or anaphylactic reactions to biologic agents or known allergy to any component of the IP formulation.
17. History of recurrent significant infections (e.g., requiring hospitalization or IV antibiotics).
18. Within 2 weeks prior to the screening visit: clinically significant active infection requiring antimicrobial medication but allowing chronic nail infections.
19. Unresected thymoma (Note: subjects with a benign thymoma resected >1 year prior to screening may enroll. Benign is defined as no known metastases and no extension into or beyond the capsule on pathological examination. Imaging to evaluate for thymoma must have been performed prior to randomization per standard of care).
20. History of cancer, except for the following:
   a. In situ carcinoma of the cervix treated with apparent success with curative therapy for >12 months prior to screening
   b. Cutaneous basal cell or squamous cell carcinoma treated with apparent success with curative therapy for >12 months prior to screening
   c. Prostate cancer treated with radical prostatectomy or radiation therapy with curative intent >3 years prior to screening and without known recurrence or current treatment
   d. Malignant thymoma resected >5 years prior to screening with no evidence of active disease and no therapy received over the previous 5 years.
21. Spontaneous or induced abortion, still or live birth, or pregnancy ≤4 weeks prior to screening.
22. Any of the following laboratory abnormalities at screening (one repeat test may be conducted to confirm results prior to randomization within the same screening period):
   a. Elevated liver enzymes (aspartate aminotransferase (AST) or alanine aminotransferase (ALT)>2.5×upper limit of normal (ULN)).
   b. Total bilirubin>1.5×ULN (unless due to Gilbert's syndrome)
   c. Estimated glomerular filtration rate (eGFR)<45 mL/min/1.73 m²
   d. CD19+ B-cell count<40 cells/μL
   e. Absolute neutrophil count (ANC)<1.2×10′ cells/μl
   f. Platelet count<75,000/μL (or <75×10⁹/L)
   g. Hemoglobin<8.0 g/dL
   h. Total immunoglobulin<600 mg/dL
23. Positive test for chronic hepatitis B infection at screening, defined as either (1) positive hepatitis B surface antigen (HBsAg) or (2) a positive hepatitis B core antibody (anti-HBc) PLUS negative hepatitis B surface antibody (anti-HBs).
   Note: Subjects with a positive anti-HBs only, or a positive anti-HBc plus positive anti-HBs and negative HBsAg, are eligible to enroll.
24. Positive test for hepatitis C virus antibody.
25. Positive HIV test.
26. Blood transfusion within 4 weeks prior to screening or during the screening period.
27. Inability to read.
28. History of active or latent tuberculosis (TB), or a positive QuantiFERON®-TB Gold test at screening, unless treatment for tuberculosis was completed per local guidelines. Subjects with latent TB or a positive QuantiFERON®-TB Gold test who are actively on anti-TB treatment can enroll if they have completed at least 1 month of anti-TB treatment and intend to complete the full course of anti-TB treatment. Subjects with an indeterminate QuantiFERON®-TB Gold test result can enroll if a repeat QuantiFERON®-TB Gold is negative or a tuberculin skin test is negative.

### Open-Label Period

Table 1 shows all procedures to be conducted during the OLP.

The OLP assessments are the same for AChR-Ab+ and MuSK-Ab+ subjects. Subjects who complete the RCP have the option to enroll in the OLP study. Subjects who discontinue the RCP early are not eligible to enter the OLP. Informed consent for OLP participation must be obtained at the time of OLP entry.

Whenever possible, OLP Day 1 should be the same day as the final day of the RCP. However, OLP Day 1 may be delayed for up to 14 days. Most procedures done at the final RCP visit do not need to be repeated at the OLP Day 1 visit if it is done within 14 days (see Table 1, footnote b). Subjects are not permitted to enter the OLP after 14 days after the final RCP visit unless there is a compelling reason that is discussed with and agreed to by the medical monitor.

The OLP includes IP infusion on OLP Day 1, Day 15, and Day 183. All subjects receive inebilizumab on OLP Days 1 and 183. On OLP Day 15, subjects who received inebilizumab in the RCP will receive placebo and subjects who received placebo in the RCP will receive inebilizumab. This allows subjects who are newly initiating inebilizumab to receive the full 2-dose initial treatment course while maintaining blinding of the RCP treatment assignment.

Subjects will continue to be followed for an additional 12 months after the last dose of inebilizumab at OLP Day 183 (until OLP Day 547). A dose will not be given at OLP Day 365. The reason to not give a dose at this timepoint is to observe whether subjects' MG remains stable once the pharmacodynamic effect wears off in subjects who have completed at least 1 year of inebilizumab treatment. These data are helpful in assessing the durability of any observed treatment response and the need for re-treatment.

It is recommended to taper non-steroidal ISTs (e.g., azathioprine, mycophenolate mofetil, mycophenolic acid) per standard of care. Likewise, for subjects on tacrolimus (allowed in Japan only), taper of tacrolimus is recommended. The rationale for tapering non-steroidal ISTs in the OLP is to reduce potential risks associated with long-term use of multiple ISTs.

Corticosteroids can either be continued at a dose of prednisone 5 mg daily or can be tapered at the discretion of the Investigator. If a subject receives a dose of prednisone that is >10 mg daily for >8 consecutive weeks in the OLP, then IP must be discontinued. The rationale for this requirement is to reduce the potential risk of adverse effects associated with long-term combination of moderate- or high-dose corticosteroids and inebilizumab.

TABLE 1
Open-Label Period Procedures for AChR-Ab+ and MuSK-Ab+ Populations

| | Study Period | | | | | | | |
|---|---|---|---|---|---|---|---|---|
| | OLP | | | | | | | |
| | OLP week | | | | | | | |
| | 0 | 2 | 13 | 26 | 39 | 52 | 65 | 78 |
| | OLP Study Day | | | | | | | |
| | | | | | | | | Day |
| | | Day | Day | Day | Day | Day | Day | 547 ± |
| | Day | 15 ± | 92 ± | 183 ± | 275 ± | 365 ± | 456 ± | 7 d or |
| | 1^{a} | 2 d | 7 d | 7 d | 7 d | 7 d | 7 d | EDV |
| Verify eligibility criteria | X | | | | | | | |
| MGQOL-15 r score; | X ^{b} | | X | X | X | X | X | X |
| PGIC; Neuro-QoL | | | | | | | | |
| C-SSRS (Since Last Visit | X ^{b} | X | X | X | X | X | X | X |
| version) | | | | | | | | |
| Weight | X ^{b} | | | | | X | | |
| Vital signs | X | X | X | X | X | X | X | X |
| Concomitant medications | X | X | X | X | X | X | X | X |
| Record healthcare resource | X | X | X | X | X | X | X | X |
| utilization | | | | | | | | |
| MG-ADL score | X ^{b} | X | X | X | X | X | X | X |
| QMG score ^{c} | X ^{b} | | X | X | X | X | X | X |
| MGC score ^{c} | X ^{b} | | X | X | X | X | X | X |
| MGFA PIS | X ^{b} | | X | X | X | X | X | X |
| MGFA clinical | X ^{b} | | | X | | X | | X |
| classification | | | | | | | | |
| Physical examination | X ^{b} | | X | X | X | X | X | X |
| Collect blood for: ^{d} | | | | | | | | |
| Hematology and serum | X ^{b} | X | X | X | X | X | X | X |
| chemistry | | | | | | | | |
| Inebilizumab ADA | X ^{b} | | X | X | X | X | | X |
| (serum) | | | | | | | | |
| Whole blood for flow | X ^{b} | | | X | | X | X | X |
| cytometry | | | | | | | | |
| Total Ig, IgM, IgG, IgA | X ^{b} | | | X | | X | | X |
| AChR or MuSK antibody | X ^{b} | | X | X | | X | | X |
| titer ^{e, f} | | | | | | | | |
| Serum biomarkers | X ^{b} | | X | X | X | X | | X |
| Plasma biomarkers ^{f} | X ^{b} | | X | X | X | X | | X |
| RNA PAXgene tube ^{f} | X ^{b} | | | X | | X | X | X |
| B-cell repertoire profiling | X ^{b} | | X ^{g} | X | | X | | X |
| (sub study in selected | | | | | | | | |
| countries) | | | | | | | | |
| Urine pregnancy test ^{h} | X | X | | X | | | | |
| Investigational product | X | X | | X | | | | |
| administration | | | | | | | | |
| Assessment of AEs/SAEs | X | X | X | X | X | X | X | X |
| Corticosteroid dispense ⁱ | X | | X | X | X | X | X | |

AChR = Acetylcholine receptor;
ADA = anti-drug antibody(ies);
AE = adverse event;
C-SSRS = Columbia-Suicide Severity Rating Scale;
d = Day;
ECG = electrocardiogram;
EDV = Early Discontinuation Visit;
Ig = immunoglobulin;
IP = Investigational product;
MG-ADL = Myasthenia Gravis Activities of Daily Living;
MGC = Myasthenia Gravis Composite;
MGFA = Myasthenia Gravis Foundation of America;
MGQOL-15r = Myasthenia Gravis Quality of Life-15, revised MuSK = Muscle-specific Kinase;
Neuro-QoL = Quality of Life in Neurological Disorders Fatigue Score;
OLP = Open-Label Period;
PGIC = Patient Global Impression of Change;
PIS = Post-intervention status;
QMG = Quantitative Myasthenia Gravis;
RNA = Ribonucleic acid;
SAE = Serious adverse event.
Note:
On days of IP administration, all procedures and blood sampling must be collected before IP administration.
^{a}For subjects choosing to continue into the Open-Label Period, end of RCP visit procedures need to be completed prior to starting any procedures or doses of the first day of OLP.
^{b} This procedure does not need to be repeated on OLP Day 1 if it was completed at the final RCP visit and the OLP Day 1 visit is done within 14 days of the final RCP visit.
^{c} Assessment should be performed at approximately the same time of day for all visits and, if possible, completed by the same assessor. Cholinesterase inhibitors (if taken) need to be held for 12 hours prior to the assessments.
^{d} Fasting is not required.
^{e} AChR antibody titer is performed for AChR-Ab+ population only; MuSK antibody titer is performed for the MuSK-Ab+ population only
^{f} Not done in China
^{g} Day 92 sample collection for MuSK-Ab+ population only.
^{h} Females of childbearing potential only; must be found negative before investigational product is administered.
ⁱ For subject on corticosteroids.

### Efficacy Assessments

### Role of the Independent Rater

Two efficacy outcomes, the QMG and the MGC, are assessed by an independent rater. The MG-ADL is recorded by an independent rater by asking questions of the subject.

The independent rater who performs the physical examination portion of the MGC should be a physician, physician-assistant, nurse practitioner, physical therapist, or other healthcare provider who is experienced with neurological examination. The QMG can be performed by any independent rater who is experienced in performing the QMG and has completed the protocol training on QMG performance. To reduce the risk of bias, an independent rater should not otherwise be involved in the subject's care, either as the site Investigator/Sub-Investigator or primary study coordinator. Whenever possible, the same independent rater should be used for a subject throughout the study. A primary and backup rater must be identified for each subject, although they need not be the same for all subjects at a site. Assessments should be done at approximately the same time of day for all visits to reduce the impact of diurnal variability of symptoms on the assessments. Cholinesterase inhibitors (if taken) should be held (i.e., should not be taken) for 12 hours prior to QMG and MGC assessments. If cholinesterase inhibitors were not held for 12 hours prior to evaluation, the evaluation should proceed, nonetheless. The time of assessment and time of last cholinesterase inhibitor use is recorded in the electronic Subject Reported Outcome (ePRO) system.

### Myasthenia Gravis Activities of Daily Living

The MG-ADL is recorded by an independent rater based on questions answered by the subject. The MG ADL is a validated measure that requires no equipment and that can be administered in 10 minutes (Wolfe G I, Barohn R J, Foster B M, Jackson C E, Kissel J T, Day J W, et al; Myasthenia Gravis-IVIG Study Group. Randomized, controlled trial of intravenous immunoglobulin in myasthenia gravis. Muscle Nerve 2002; 26:549-52). MG-ADL is an 8-item questionnaire that focuses on relevant symptoms and functional performance of activities of daily living in subjects with MG over the previous 7 days. The MG-ADL assesses disability secondary to ocular (2 items), bulbar (3 items), respiratory (1 item), and gross motor or limb (2 items) impairment related to effects from MG. In this functional status instrument, each response is graded 0 (normal) to 3 (most severe). The range of total MG-ADL scores is 0-24. The minimal important difference for this PRO questionnaire is a 2-point improvement (Barnett C, Herbelin L, Dimachkie M M, Barohn R J. Measuring clinical treatment response in myasthenia gravis. Neurol Clin 2018; 36:339-53). The MG-ADL is recorded in the ePRO system.

### Myasthenia Gravis Quality of Life-15, Revised

MGQOL-15r is a PRO and does not require an independent rater. MGQOL-15r is a validated, subject-scored instrument, which measures the impact of MG on health-related quality of life (HRQoL) (Burns T M, Conaway M R, Cutter G R, Sanders D B, Muscle Study Group. Less is more, or almost as much: a 15-item quality-of-life instrument for myasthenia gravis. Muscle Nerve 2008; 38:957-63). The 15 items in the questionnaire evaluate mobility (9 items), symptoms (3 items), general contentment (1 item), and emotional well-being (2 items) domains. Each item is rated on a 3-point scale ranging from 0 (“not at all”) to 2 (“very much”) based on their experience “over the past few weeks.” Item scores are summed to generate a total score ranging from 0-30, with higher scores indicating worse HRQoL. The MGQOL-15r is recorded in the ePRO system.

### Subject Global Impression of Change

The PGIC is a subject-reported, 7-point scale that evaluates whether there has been an improvement or decline in the subject's disease-related status. The PGIC is recorded in the ePRO system.

### Neuro-QoL Fatigue Score

The Neuro-QoL is a collection of 13 subscales that measure HRQoL in adults and children with neurological disorders (National Institute of Neurological Disorders and Stroke [NINDS, 2019]). The Neuro-QoL scales were developed with subject, caregiver, and expert input, and have been rigorously tested for validity, reliability, and responsiveness (Cella D, Lai J S, Nowinski C J, Victorson D, Peterman A, Miller D, et al. Neuro-QOL: brief measures of health-related quality of life for clinical research in neurology. Neurology 2012; 78:1860-7). Fatigue, a commonly reported symptom in MG, is measured with the Neuro-QoL in this study.

The Neuro-QoL Fatigue subscale is a 19-item, self-assessment of fatigue that incorporates “sensations ranging from tiredness to an overwhelming, debilitating, and sustained sense of exhaustion that decrease one's capacity for physical, functional, social, and mental activities” (National Institute of Neurological Disorders (NINDS). User manual for the quality of life in neurological disorders (Neuro-QoL) measures, version 2.0. March 2015. Accessed Dec. 9, 2019, from http://www.healthmeasures. net/images/neuro_qol/Neuro-QOL_User_Manual_v2_24Mar 2015.pdf). The range of potential total scores is 19-95. Higher scores indicate greater fatigue. Neuro-QoL was used in a study to assess fatigue in subjects with AChR-Ab+MG (Andersen H, Mantegazza R, Wang J J, O'Brien F, Patra K, Howard J F Jr; REGAIN Study Group. Eculizumab improves fatigue in refractory generalized myasthenia gravis. Qual Life Res. 2019; 28:2247-54). The association between Neuro-QoL and MG-ADL, QMG, and MG-QOL15 scales was also measured, and showed a consistently strong, positive correlation with improvements in MG specific outcome measures, see Table 2. The Neuro-QOL is recorded in the ePRO system.

TABLE 2
Outcome Measures

| | | Patient- | | | |
|---|---|---|---|---|---|
| | Total | reported | Score | | |
| | items | items | range | MID | Interpretation |
| MG-ADL | 8 | 8 | 0-24 | 2 points | Lower score, |
| | | | | | less severe |
| | | | | | disease |
| QMG | 13 | 0 | 0-39 | 3 points | Lower score, |
| | | | | | less severe |
| | | | | | disease |
| MGC | 10 | 4 | 0-50 | 3 points | Lower score, |
| | | | | | less severe |
| | | | | | disease |
| MG- | 15 | 15 | 0-30 | NA | Lower score, |
| QOL-15r | | | | | better HRQOL |

HRQOL, health-related quality of life;
MG-ADL, myasthenia gravis activities of daily living;
MGC, myasthenia gravis composite;
MG-QOL-15r, myasthenia gravis quality of life 15 revised;
MID, minimal important difference;
NA, not available;
QMG, quantitative myasthenia gravis.

### Quantitative Myasthenia Gravis Score

The QMG score is determined by an independent rater. The QMG is a validated outcome comprised of 13 items: ocular (2 items), facial (1 item), bulbar (2 items), gross motor (6 items), axial (1 item), and respiratory (1 item). Each item has a possible score of between 0 and 3 points. The total score range is 0-39 points, with a higher score indicating more severe disease. A 3-point improvement in the QMG score is considered clinically meaningful (Barohn R, McIntire D, Herbelin L, Wolfe G, Nations S, Bryan W. Reliability testing of the quantitative myasthenia gravis score. Ann N Y Acad Sci 1998; 841:769-72; Zinman L, Baryshnik D, Bril V. Surrogate therapeutic outcome measures in subjects with myasthenia gravis. Muscle Nerve 2008; 37:172-6). The QMG score is recorded in the ePRO system.

### Myasthenia Gravis Composite Score

The MGC score is determined by an independent rater. The MGC consists of test items from the MG-ADL and the QMG that measure symptoms and signs of MG, with weighted response options (Burns T M, Conaway M, Sanders D B; MG Composite and MG-QOL15 Study Group. The MG Composite: a valid and reliable outcome measure for myasthenia gravis. Neurology 2010; 74:1434-40). Scores range from 0-50, with higher scores indicating worse disease manifestations. A 3-point improvement in score has been shown to correlate with improvement that is meaningful to the subject (Burns T M. The MG composite: an outcome measure for myasthenia gravis for use in clinical trials and everyday practice. Ann N Y Acad Sci 2012; 1274:99-106). The independent rater determines the MGC score based on elements of the QMG, elements of the MG-manual muscle testing (MG-MMT), and subject responses for the MG ADL for that visit. The MGC score is recorded in the ePRO system.

### Myasthenia Gravis Foundation of America Post-Intervention Status

MGFA PIS is a system for describing the clinical status of subjects with MG at any time after initiation of treatment (Jaretzki A, Barohn R, Emstoff R, Kaminski H, Keesey J, Penn A, et al. Myasthenia gravis: recommendations for clinical research standards. Task Force of the Medical Scientific Advisory Board of the Myasthenia Gravis Foundation of America. Neurology 2000; 55:16-23). The Investigator/sub-Investigator assesses whether or not the subject has achieved ‘minimal manifestation status’ (defined as no symptoms or functional limitations from MG but could have some muscle weakness) (Sanders D B, Wolfe G I, Benatar M, Evoli A, Gilhus N E, Illa I, et al. International consensus guidance for management of myasthenia gravis. Executive Summary. Neurology 2016; 87:419-25) and this is recorded in the ePRO system. Change in clinical status (Improved, Unchanged, Worse) is calculated automatically based on the subject's MGC score and will not require specific input from the site. MGC was selected over QMG to determine improvement and worsening because it is weighted for clinical significance and incorporates PROs (Benatar M, Sanders D B, Burns T M, Cutter G R, Guptill G T, Baggi F, et al. Recommendations for myasthenia gravis clinical trials. Muscle Nerve 2012; 45:909-17). ‘Improved’ status is defined as a >3-point decrease and ‘Worse’ status is defined as a >3-point increase in MGC from baseline MGC score. ‘Unchanged’ status is defined as <3-point change from baseline.

### Healthcare Resource Utilization

Healthcare resource utilization is reported by the subject and recorded by the study coordinator. The following variables are collected: days of hospitalization (general ward and intensive care ward), emergency department visits, number of IVIg treatments, and number of PLEX treatments since the last study visit. Healthcare resource utilization is recorded in the eCRF.

### INCORPORATION BY REFERENCE

This patent application incorporates by reference in their entireties for all purposes the following patent publications and applications: Int'l Appl. Nos. PCT/US2020/29613 and PCT/US2007/077916.

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A method of treating myasthenia gravis (MG), the method comprising administering to a subject in need of treatment for MG an antibody comprising: Complementarity-Determining Regions (CDRs) HCDR1, HCDR2, HCDR3, LCDR1, LCDR2, and LCDR3, wherein the HCDR1, HCDR2, HCDR3, LCDR1, LCDR2, and LCDR3 comprise the amino acid sequences of SEQ ID NOs: 3, 4, 5, 6, 7 and 8, respectively, and wherein the antibody is administered intravenously at a dose of from about 250 mg to about 350 mg every 6 months, thereby treating the MG.

2. The method of claim 1, further comprising a first dose of 300 mg, a second dose of 300 mg two weeks after the first dose, and subsequent doses of 300 mg every 6 months following the first initial dose.

3. (canceled)

4. The method of claim 1, wherein the administering is effective in
(i) depleting at least about 90% of circulating CD20+ B cells for at least six months;
(ii) does not increase incidence of an infection in the subject; or
(iii) (i) and (ii).

5. The method of claim 4, wherein the administering reduces a level of peripheral blood CD20⁻ plasmablasts and plasma cells within about 8 days following the administering.

6. The method of claim 1, wherein the dose is about 300 mg.

7. The method of claim 1, wherein the administering is effective in reducing MG-related disability.

8. The method of claim 1, wherein the administering is effective in reducing frequency of MG exacerbations.

9. The method of claim 1, wherein the subject is acetylcholine receptor antibody positive (AChR-Ab+).

10. The method of claim 1, wherein the subject is muscle-specific kinase-antibody positive (MuSK-Ab+).

11. The method of claim 1, wherein the subject is acetylcholine receptor antibody positive (AChR-Ab+) and muscle-specific kinase-antibody positive (MuSK-Ab+).

12. The method of claim 1, wherein the antibody comprises a heavy chain variable region (VH) comprising the amino acid of SEQ ID NO:1, and a light chain variable region (VL) comprising the amino acid of SEQ ID NO: 2.

13. The method of claim 1, wherein the antibody comprises a heavy chain comprising the amino acid of SE ID NO: 9; and a light chain comprising the amino acid of SEQ ID NO: 10.

14. The method of claim 1, wherein the subject is further administered one or more additional therapies.

15. The method of claim 14, wherein the one or more additional therapies comprise one or more standard-of-care therapies.

16. The method of claim 15, wherein the one or more standard-of-care therapies comprises a corticosteroid, a nonsteroidal immunosuppressive therapy, or both.

17. The method of claim 16, wherein the one or more standard-of-care therapies comprises the corticosteroid, and wherein the corticosteroid comprises prednisone.

18. The method of claim 17, wherein the one or more additional therapies are one or more of azathioprine, mycophenolate mofetil, mycophenolic acid, or tacrolimus.

19. The method of claim 1, wherein the antibody is Inebilizumab.

20. (canceled)

21. The method of claim 1, wherein the administering treats the MG as determined by:
a) a reduction in MG activities of daily living score;
b) a reduction of a MG score;
c) an increase in a quality-of-life score; and/or
d) a reduction in incidence of exacerbations.

22. The method of claim 1, wherein the MG is refractory MG.

23. The method of claim 1, wherein the subject in need of treatment for MG has uncontrolled MG, on a standard-of-care therapy, as determined by one or more of:
a) a MG Foundation of America classification of II, III, or IV;
b) an MG activities of daily living (MG-ADL) score≥6, with >50% of this score being attributed to non-ocular items; or
c) a quantitative MG (QMG) score≥11.

24. The method of claim 17, wherein if the subject in need of treatment for MG is receiving a dosage of prednisone over 5 mg/day or equivalent, the subject will undergo tapering of the prednisone dosage to 5 mg/day.

25. The method of claim 18, comprising a maximum dosage of:
a) tacrolimus≤3 mg/day;
b) azathioprine≤3 mg/kg/day;
c) mycophenolate mofetil≤3 g/day; and/or
d) mycophenolic acid≤1440 mg/day.

26. The method of claim 23, wherein the subject in need of treatment for MG has uncontrolled MG, on a standard-of-care therapy, as determined by the MG-ADL score of ≥6, with >50% of the score being attributed to non-ocular items, and wherein the MG-ADK score is reduced after the administering.

27. (canceled)

28. The method of claim 1, comprising determining:
a) a B-cell subset phenotype;
b) a B-cell receptor repertoire;
c) a B-cell gene expression profiling; or
d) any combination thereof.

29. The method of claim 1, wherein the administering is effective in reducing or eliminating mature plasma cells in the subject.

30. The method of claim 1, wherein the administering results in longer-lasting B-cell reduction or elimination as compared to an otherwise comparable method comprising administration of an anti-CD20 therapy.

31-42. (canceled)

43. A method of treating myasthenia gravis (MG), the method comprising administering to a subject in need of treatment for MG an antibody that comprises a heavy chain variable region (VH) comprising the amino acid of SEQ ID NO:1, and a light chain variable region (VL) comprising the amino acid of SEQ ID NO: 2, wherein the antibody is administered intravenously at a dose of 300 mg every 6 months, thereby treating the MG.

44. The method of claim 43, further comprising a first dose of 300 mg, a second dose of 300 mg two weeks after the first dose, and subsequent doses of the 300 mg every 6 months following the first initial dose.

45. The method of claim 43, wherein a subject that is acetylcholine receptor antibody positive (AChR-Ab+) and/or muscle-specific kinase-antibody positive (MuSK-Ab+) is administered the antibody.


SEQUENCE LISTING (10 sequences)

SEQ ID NO: 1 - protein, 121 aa, Artificial Sequence
  recombinant peptide
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 2 - protein, 111 aa, Artificial Sequence
  recombinant polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIK

SEQ ID NO: 3 - protein, 5 aa, Artificial Sequence
  Synthetic peptide
SSWMN

SEQ ID NO: 4 - protein, 17 aa, Artificial Sequence
  Synthetic peptide
RIYPGDGDTNYNVKFKG

SEQ ID NO: 5 - protein, 12 aa, Artificial Sequence
  Synthetic peptide
SGFITTVRDFDY

SEQ ID NO: 6 - protein, 15 aa, Artificial Sequence
  Synthetic peptide
RASESVDTFGISFMN

SEQ ID NO: 7 - protein, 7 aa, Artificial Sequence
  Synthetic peptide
EASNQGS

SEQ ID NO: 8 - protein, 9 aa, Artificial Sequence
  Synthetic peptide
QQSKEVPFT

SEQ ID NO: 9 - protein, 451 aa, Artificial Sequence
  recombinant polypeptide
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, Artificial Sequence
  recombinant polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC